(19) United States
(12) Patent Application Publication
(10) Pub. No.: US 2024/0377229 A1
(43) Pub. Date: Nov. 14, 2024

(54) ENVIRONMENTAL SENSING DEVICE AND APPLICATION METHOD THEREOF

(71) Applicant: Soiltech Wireless Inc., American Falls, ID (US)

(72) Inventor: Ehsan Soltanmohammadi, American Falls, ID (US)

(73) Assignee: Soiltech Wireless Inc., American Falls, ID (US)

(21) Appl. No.: 18/648,023

(22) Filed: Apr. 26, 2024

Related U.S. Application Data
(63) Continuation-in-part of application No. 16/809,592, filed on Mar. 5, 2020.

(51) Int. Cl. G01D 11/24; A01G 7/00; G01D 21/02

(52) U.S. Cl. CPC G01D 11/245; G01D 21/02; G01N 33/245; A01G 7/00

(57) ABSTRACT

An environmental sensing device is rugged and shaped to have features that do not extend significantly from the body of the environmental sensing device thereby allowing the environmental sensing device to be buried with crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops as they are transported, remain with the crops as the crops loaded into storage units, and finally remain with the crops as the crops are stored.

### RELATED APPLICATIONS

This application is a continuation-in-part of Soltanmohammadi, U.S. patent application Ser. No. 16/809,592 (attorney docket number SOIL001), filed Mar. 5, 2020, entitled “ENVIRONMENTAL SENSING DEVICE AND APPLICATION METHOD THEREOF”, which is incorporated by reference in its entirety as if it were fully set forth herein.

### FIELD OF THE INVENTION

The present disclosure substantially relates to an environmental sensing device and application method thereof, and, more particularly, to an environmental sensing device that is specifically designed to be sufficiently rugged and self-contained to allow the same sensing device to remain with the crops and be subjected to the same processing as the crops throughout the entire crop growth and processing cycle. In other words, disclosed herein is an environmental sensing device that is specifically designed to be sufficiently rugged and self-contained so that a single environmental sensing device is capable of being planted with crops and remaining in the ground with the crops while they grow, can be harvested with the crops, can be transported with the crops, and can be stored with the crops, to collect environmental data related to the crops throughout the entire crop growth and processing cycle.

### BACKGROUND

Ideally, in order to achieve effective automated crop management, and to ensure the quality of the crops, it is highly desirable to monitor the environmental conditions of the crops during each stage of the crop processing cycle, e.g., during planting, growth, harvesting, loading, transporting, unloading, and storage of the crops.

Taking the potato crop as an example, it desirable to monitor and collect environmental data about the temperature, humidity, and moisture of the soil during the planting and growth of the potato; the environmental temperature, humidity, moisture and forces imposed on the potato during mechanical harvesting and loading of the potato into transportation vehicles; the environmental temperature, humidity, moisture, forces imposed on the potato, and the location of the potato, during transportation of the potato; the environmental temperature, humidity, moisture and forces imposed on the potato as the potato is loaded into storage units; and the environmental temperature, humidity, moisture, around the potato during storage of the potato.

In order to collect a complete set of environmental data associated with a crop, truly continuous monitoring and collection of the environmental data and parameters discussed above must be performed. Truly continuous monitoring of the crops, and collection of environmental data at all stages of crop processing would provide a complete set of data regarding the processing of the crops that, if available, would be extremely helpful to properly manage the crop processing cycle, to avoid crop diseases and ensure the quality and the yield of the crop. Additionally, if it were possible to continuously and accurately monitor and collect complete environmental data during other crop processing stages, such as harvesting, transport or storage, etc., of the crops, the crops could be made less susceptible to damage, disease, or spoilage and waste.

Consequently, if accurate and complete environmental data were readily available while the crop was being grown, harvested, loaded, transported, unloaded, and stored, truly effective analysis of the farming process at all stages could be obtained and truly effective automated crop management would be possible.

In addition, as Machine Learning (ML) and Artificial Intelligence (AI) become the tools of choice to automate and perfect farming techniques, very detailed and complete environmental data sets indicating the temperature, humidity and moisture of the soil during the planting and growth of the crop; the environmental temperature, humidity, moisture and forces imposed on the crop during mechanical harvesting and loading of the crop into transportation vehicles; the environmental temperature, humidity, moisture, forces imposed on the crop, and the location of the crop, during transportation of the crop; the environmental temperature, humidity, moisture and forces imposed on the crop as the crop is loaded into storage units, and the environmental temperature, humidity, moisture, around the crop during storage of the crop is highly desirable and/or required. This is because the more detailed and complete the data used to train and update the models used by ML and AI systems, the better these models become at predicting and managing the entire crop processing cycle.

In the prior art, attempts to collect at least part of the desired environmental information involved using prior art environmental sensing devices, also referred to herein as simply “currently available sensing devices” or “conventional sensing devices.” However, as discussed below, currently available sensing devices are not suitable for the type of continuous monitoring and environmental data collection including: the temperature, humidity, and moisture of the soil during the growth of the crop; the environmental temperature, humidity, moisture, and forces imposed on the crop during mechanical harvesting and loading of the crop into transportation vehicles; the environmental temperature, humidity, moisture, forces imposed on the crop, and the location of the crop, during transportation of the crop; the environmental temperature, humidity, moisture, and forces imposed on the crop as the crop is loaded into storage units, and the environmental temperature, humidity, moisture, around the crop during storage of the crop.

This is because, as discussed below, currently available sensing devices are not designed to stay with the crops during the entire processing cycle of the crops, i.e., currently available sensing devices are not designed to remain with the crops and be subjected to the same processing as the crops, throughout the entire growth and processing cycle.

As an example, many currently available sensing devices perform measurements according to osmotic balance. However, these conventional moisture sensing devices used with prior art sensing devices are often used without any self-contained, waterproof, and rugged, protective case, so the measurement accuracy is easily affected by environmental factors and forces.

In addition, prior art sensing devices usually include a processor and at least one probe electrically connected to the processor by a wire or a cable. The probe of these prior art sensing devices are typically inserted into soil and the antenna/processor part of the wire would be exposed to air for farmers to read the measurement.

In other cases, the prior art sensing devices are rod shaped to allow for data collection at various “levels” and/or to accommodate probes or sensors that extend above the surface level of the crops.

Consequently, prior art sensing devices are easily damaged by other farming and or processing steps such as harvesting, loading, unloading etc. Consequently these prior art sensing devices need to be removed from the field before harvesting, otherwise prior art sensing device may interfere, or can be damaged by, the harvesting process. Hence, the environmental conditions may not be fully or continuously monitored, nor can environmental data be collected, during the harvesting, loading/unloading, transportation or storage stages using these prior art sensing devices.

Therefore, how to monitor the environmental conditions (e.g. soil moisture and/or humidity, etc.) and continuously collect environmental data with reliability and accuracy during all the different stages (e.g. planting, growth, harvesting, storage or transportation, etc.) of the crop using a single environmental sensing device that remains with the crops throughout the entire crop growth and processing cycle has become an urgent problem to be solved in the farming industry.

The urgency of the problem has become even more pronounced lately since accurate and very detailed environmental data collected on a continuous basis while the crop was being grown, harvested, transported, and stored, is required to effectively train models required to employ ML and AI systems for truly effective, real time, and predictive, automated farm and crop management.

However, as noted, currently available sensing devices are not suitable for the type of continuous monitoring desired and needed to train AI and ML models. This is because, as noted, currently available sensing devices are not designed to stay with the crops during the entire lifetime of the crops, i.e., currently available sensing devices have structures and features that prevent a given currently available sensing device from remaining with the crops and being subjected to the same processing as the crops, throughout the entire growth and processing cycle.

As an example, while some currently available sensing devices can be planted with, or stored with, the crops. However, these currently available sensing devices are either rod-shaped, i.e., have a length dimension that is significantly larger that their width dimension, or have external features, such as the probes discussed above or data transmission antennas, that extend well beyond the exterior surface of the currently available sensing devices. As a result, while some currently available sensing devices can be planted or stored with the crops, they cannot be harvested with the crops using standard mechanical/automated harvesting machines or loaded and unloaded with the crops because their shape and/or external features make them too delicate for harvesting or loading/unloading using mechanical/automated harvesting and/or loading/unloading machines.

In addition, some currently available sensing devices are designed to be stored with the crops. However, these currently available sensing devices are again typically either rod-shaped, are often quite large, and/or have external features that extend well beyond the exterior surface of the currently available sensing devices. As a result, while some currently available sensing devices can be stored with the crops, they cannot be harvested, loaded, and unloaded with the crops using standard mechanical/automated harvesting or loading/unloading procedures and machines, nor can they remain with the crops as they are transported and/or loaded and unloaded into storage units because their shape and/or external features make them too delicate for transportation and loading.

In addition, some currently available sensing devices are designed to be only partially buried with the crops and require that at least a portion of the sensing device remains above the soil surface, typically in order to transmit data. These currently available sensing devices therefore are not actually fully buried with the crop. This not only adversely effects the ability of these currently available sensing devices to accurately collect data at the depth of the crop, but these currently available sensing devices are again typically either rod-shaped, i.e., have a length dimension that is significantly larger that their width dimension, are often quite large, and/or have external features that extend well beyond the exterior surface of the currently available sensing devices. Therefore, these currently available sensing devices cannot be harvested with the crops using standard mechanical/automated harvesting machines because their shape and/or external features make them too delicate for harvesting using mechanical/automated harvesting machines. In addition, these currently available sensing devices are even more susceptible to damage and environment forces even before harvesting due to the fact that portions of these currently available sensing devices extend above the surface of the soil at all times.

Finally, many currently available sensing devices are not designed to be buried with the crops, or collect data from a buried position, at all. Typically, these currently available sensing devices are mounted on a base platform, such as a wheel, sled, or support structure that is then rolled, slid, or hovered over the soil surface to collect data about the soil in which the crops are buried. Obviously, these currently available sensing devices are not designed to, and cannot, stay with the crops during the entire lifetime of the crops, i.e., they cannot remain with the crops, and be subjected to the same processing as the crops, throughout the entire growth and processing cycle.

As a result, none of the currently available sensing devices is designed to stay with the crops and continuously collect environmental data about the crops during the entire lifetime of the crops, i.e., currently available sensing devices cannot be buried with crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops are they are transported, remain with the crops as the crops unloaded into storage units, and finally remain with the crops as the crops are stored.

As a result, individual currently available sensing devices are not capable of collecting data regarding the crops at every stage of the growth and processing of the crops. Therefore, using currently available sensing devices the type of accurate and complete data required to effectively monitor and automate crop farming is not obtained. In addition, the detailed, complete, and continuous data needed to effectively train ML and AI models cannot be obtained using currently available sensing devices.

Consequently, there is a long-standing, and more recently even more urgent, technical problem in the crop growing industry of an inability to accurately collect complete and accurate data regarding the environmental factors and forces a given crop is subjected to at each stage of the lifetime of the crops using a single unit or type of environmental sensing device.

What is needed is a method and system that provides an environmental sensing device that can be buried with the crops and stay with the crops during the entire lifetime of the crops, i.e., can be buried with crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops as they are transported, and remain with the crops as the crops unloaded into storage units, and finally remain with the crops as the crops are stored.

### SUMMARY

Disclosed herein are various embodiments of a method and system that provides a technical solution to the long-standing technical problem of accurately collecting environmental data regarding the environmental factors and forces a given crop is subjected to at each stage in the lifetime of the crops using a single unit or type of environmental sensing device.

To this end, disclosed herein is a method and system that provides an environmental sensing device that can be buried with the crops and stay with the crops during the entire lifetime of the crops, i.e., an environmental sensing device that can be buried with crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops are they are transported, remain with the crops as the crops loaded into storage units, and finally remain with the crops as the crops are stored.

In one embodiment, an environmental sensing device includes an environmental sensing device body that forms an environmental sensing device interior space, also referred to herein as an environmental sensing device accommodating space, and an environmental sensing device exterior surface. In one embodiment, the environmental sensing device interior/accommodating space is protected from the exterior environment by the environmental sensing device exterior surface such that the environmental sensing device interior/accommodating space is moisture resistant or moisture proof.

In various embodiments, the environmental sensing device body can be formed of Acrylonitrile Butadiene Styrene and polycarbonate (ABS+PC), High Density Polyethylene (HDPE). In some embodiments, the environmental sensing device body can be formed using injection molding and PCBA. In other embodiments, the environmental sensing device body can be formed of any material discussed herein, and/or as available/known in the art at the time of filing, and/or as made available/becomes known after the time filing capable of being buried, harvested, loaded/unloaded, transported and stored with crops.

In one embodiment, the environmental sensing device body is formed in a substantially cylindrical shape with a substantially oval or elliptical cross section wherein a length dimension of the environmental sensing device body is no more than twice a width, or largest diameter, dimension of the environmental sensing device.

In one embodiment, the environmental sensing device body is formed in a substantially cylindrical shape with a substantially circular cross section wherein a length dimension of the environmental sensing device body is no more than twice a width, or diameter, dimension of the environmental sensing device.

In one embodiment, the environmental sensing device body is formed in a substantially ellipsoidal shape.

In one embodiment, the environmental sensing device body is formed in a substantially cube shape.

In one embodiment, the environmental sensing device body is formed in a substantially cuboid shape wherein a length dimension of the environmental sensing device body is no more than twice a width dimension of the environmental sensing device.

In one embodiment, the environmental sensing device body is formed in a substantially spherical shape.

In one embodiment, the environmental sensing device body is formed in a substantially multi-faced shape having a multi-side cross section, such as a triangular, hexagonal, octanal, etc., cross section, wherein a length dimension of the environmental sensing device body is no more than twice a width dimension of the environmental sensing device.

In one embodiment, the environmental sensing device body is formed to have dimensions, e.g., length, width, girth, volume and/or weight, similar to that of an average individual unit of the crop with which it is to be used.

In one embodiment, the environmental sensing device body includes at least one first conducting unit and at least one second conducting unit attached/on or formed into at least part of the exterior surface of the environmental sensing device body.

In one embodiment, the at least one first conducting unit and at least one second conducting unit are attached to the exterior surface of the environmental sensing device body such that the at least one first conducting unit and at least one second conducting unit extend no more than 3 millimeters from the exterior surface of the environmental sensing device body.

In one embodiment, the at least one first conducting unit and at least one second conducting unit are attached to the exterior surface of the environmental sensing device body such that the at least one first conducting unit and at least one second conducting unit extend no more than 2 millimeters from the exterior surface of the environmental sensing device body.

In one embodiment, the at least one first conducting unit and at least one second conducting unit are attached to the exterior surface of the environmental sensing device body such that the at least one first conducting unit and at least one second conducting unit extend no more than 1 millimeter from the exterior surface of the environmental sensing device body.

In one embodiment, the at least one first conducting unit and at least one second conducting unit are recessed in the exterior surface of the environmental sensing device body such that the at least one first conducting unit and at least one second conducting unit do not extend beyond the exterior surface of the environmental sensing device body at all and are substantially flush with the exterior surface of the environmental sensing device body.

In one embodiment, the at least one first conducting unit and at least one second conducting unit are recessed in the exterior surface of the environmental sensing device body such that the at least one first conducting unit and at least one second conducting unit are recessed below the exterior surface of the environmental sensing device body.

In one embodiment, the first conducting unit and the second conducting unit are annular and disposed around the exterior surface of the environmental sensing device body. In one embodiment, the first conducting unit and the second conducting unit are annular and disposed around the exterior surface of the environmental sensing device body in recessed regions formed in the exterior surface of the environmental sensing device body.

In one embodiment, the first conducting unit and the second conducting unit are made of stainless steel, titanium, plated gold, of any material discussed herein, and/or as available/known in the art at the time of filing, and/or as made available/becomes known after the time filing that is a conductor and corrosion resistant and is capable of being buried, harvested, loaded/unloaded, transported and stored with crops.

In one embodiment, the disclosed environmental sensing device includes an electric measuring unit, a processing unit, and a power supply unit. In one embodiment, the electric measuring unit, the processing unit, and the power supply unit are disposed entirely within the environmental sensing device interior/accommodating space.

In one embodiment, the electric measuring unit measures current, voltage, capacitance, and temperature of the surrounding environment corresponding to the first conducting unit and the second conducting unit. In one embodiment, the electric measuring unit further transmits a first signal according to the measurement of the current, voltage, capacitance, and temperature of the environment.

In one embodiment, the processing unit is disposed entirely in the environmental sensing device interior/accommodating space for receiving the first signal and calculating a moisture of the surrounding environment according to environmental data and the measurement of the current, voltage, capacitance, and temperature of the surrounding environment.

In one embodiment, the power supply unit is disposed entirely in the environmental sensing device interior/accommodating space for supplying power to the electric measuring unit and the processing unit.

In one embodiment, the disclosed environmental sensing device also includes a temperature sensing unit for sensing the temperature of the surrounding environment and transmitting a second signal.

In one embodiment, the processing unit is further used to receive the second signal and to calibrate the measurement of the temperature of the surrounding environment by the second signal. In one embodiment, the processing unit further calculates the moisture of the surrounding environment according to the environmental data, the measurement of the current, voltage, and capacitance of the surrounding environment, and the calibrated temperature of the surrounding environment.

In one embodiment, the disclosed environmental sensing device includes a communication unit disposed entirely in the environmental sensing device interior/accommodating space and electrically connected to the processing unit for communicating with an external data processing device, such as a cellphone or personal computer.

In one embodiment, the communication unit, including a data transmission device, is entirely disposed within the environmental sensing device interior/accommodating space. In various embodiments, the communication unit can be a WiFi communication unit, a LoRa communication unit, a Cellular communication unit, a Bluetooth communication unit, and/or any communication unit as discussed herein, and/or as available/known in the art at the time of filing, and/or as made available/becomes known after the time filing that does not require an antenna that extends more than 3 mm from the environmental sensing device body.

In one embodiment, the disclosed environmental sensing device also includes a positioning unit disposed in the environmental sensing device interior/accommodating space and electrically connected to the processing unit for tracking the location of the environmental sensing device.

As noted, in one embodiment, the disclosed environmental sensing device also includes a temperature sensing unit. In one embodiment, the temperature sensing unit is disposed entirely within the environmental sensing device interior/accommodating space for sensing temperature of the surrounding environment and transmitting a second signal. In one embodiment, the communication unit transmits a third signal according to the location of the environmental sensing device to a database, wherein the database pre-saves worldwide geographic information. In one embodiment, the database sends the geographic information of the location of the environmental sensing device, based on the data in the third signal, to the communication unit.

As noted, in one embodiment, the processing unit is further used to receive the second signal and calibrate the measurement of the temperature of the surrounding environment by the second signal.

In one embodiment, the processing unit then calculates the moisture of the surrounding environment according to the environmental data, the measurement of the current, voltage, and capacitance of the surrounding environment, the calibrated temperature of the surrounding environment and the geographic information.

In one embodiment, the disclosed environmental sensing device includes a storage unit disposed in the environmental sensing device interior/accommodating space for receiving and recording the measurement of the current, voltage, capacitance, and temperature of the surrounding environment, the moisture calculated by the processing unit, and the corresponding time.

In one embodiment, the disclosed environmental sensing device further includes an acceleration sensing unit disposed in the environmental sensing device interior/accommodating space and electrically connected to the processing unit for sensing external forces received by the disclosed environmental sensing device body.

In one embodiment, the disclosed environmental sensing device includes a fixing frame disposed in the environmental sensing device interior/accommodating space. In one embodiment, the fixing frame has at least one cavity for fixing components disposed in the environmental sensing device interior/accommodating space.

As discussed above, in one embodiment, the only elements of the disclosed environmental sensing device that reside outside the environmental sensing device interior/accommodating space are the at least one first conducting unit and at least one second conducting unit.

As also discussed above, in one embodiment, the at least one first conducting unit and at least one second conducting unit are attached to, or recessed in, the exterior surface of the environmental sensing device body such that the at least one first conducting unit and at least one second conducting unit extend no more than 3 millimeters from the exterior surface of the environmental sensing device body.

Consequently, in one embodiment, the disclosed environmental sensing devices have no probes or other features that extend more than 3 millimeters from the environmental sensing device body and environmental sensing device exterior surface.

In addition, in one embodiment, the disclosed environmental sensing device body is either cylindrical, cuboid, ellipsoidal, spherical, cubic, or and has a length dimension that is no more than twice a width or diameter dimension of the environmental sensing device.

As a result, the disclosed environmental sensing devices are rugged, waterproof, and have no exterior feature that extends more than 3 millimeters from the environmental sensing device body. Thus, in contrast to the prior art, the disclosed environmental sensing devices are extremely rugged and are designed to, and are capable of, being buried with the crops and staying with the crops during the entire lifetime of the crops.

Consequently, the disclosed environmental sensing devices can each be buried with the crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops are they are transported, then remain with the crops as the crops loaded into storage units, and finally remain with the crops as the crops are stored; all using a single disclosed environmental sensing device.

The present invention further provides a method of monitoring crops.

In one embodiment, the method includes providing at least one of the disclosed environmental sensing devices; burying the disclosed environmental sensing device in the soil in which crops grow; harvesting the environmental sensing device together with the crops when harvesting the crops; loading the disclosed environmental sensing device into to transportation vehicles along with the crops; transporting the disclosed environmental sensing device together with the crops when transporting the crops; unloading the disclosed environmental sensing device with the crops into storage units; and storing the disclosed environmental sensing device together with the crop when the crops are stored.

To this end, in one embodiment, a process for measuring environmental conditions includes providing at least one of the disclosed environmental sensing devices and then burying the environmental sensing device(s) in the soil where the crops grow when planting the crops.

In one embodiment, the process for measuring environmental conditions around crops further includes harvesting the environmental sensing device(s) together with the crops when harvesting the crops.

In one embodiment, the process for measuring environmental conditions around crops further includes disposing an acceleration sensing unit in at least one of the environmental sensing device(s) interior/accommodating space and electrically connecting the acceleration sensing unit to the processing unit for sensing an external force received using the body and measuring g-forces incurred during harvest and transportation by the acceleration sensing unit; and transmitting the external force and the g-force data to an external data processing device using the communication unit.

In one embodiment, the process for measuring environmental conditions around crops further includes transporting the environmental sensing device(s) together with the crops when transporting the crops.

In one embodiment, the process for measuring environmental conditions around crops further includes disposing a humidity sensing unit in at least one of the environmental sensing devices interior/accommodating space for sensing humidity of the surrounding environment of the crops.

In one embodiment, the process for measuring environmental conditions around crops further includes tracking a location of the environmental sensing device(s) and the crops using the positioning unit.

In one embodiment, the process for measuring environmental conditions around crops further includes sensing temperature of the surrounding environment of the crops using the temperature sensing unit; sensing an external force received by the body and measuring g-forces incurred during transportation by the acceleration sensing unit; and transmitting the humidity, the location, the temperature and the external force to an external data processing device by the communication unit to allow users to monitor the environmental conditions while in transit.

In one embodiment, the process for measuring environmental conditions around crops further includes storing the environmental sensing device(s) together with the crops when the crops are stored.

In one embodiment, the process for measuring environmental conditions around crops further includes sensing humidity of the surrounding environment of the crops using the humidity sensing unit.

In one embodiment, the process for measuring environmental conditions around crops further includes tracking a location of the environmental sensing device(s) and the crops using the positioning unit.

In one embodiment, the process for measuring environmental conditions around crops further includes sensing temperature of the surrounding environment of the crops using the temperature sensing unit; and transmitting the humidity, the location and the temperature to an external data processing device by the communication unit to allow users to monitor the environmental conditions while in storage.

The disclosed environmental sensing devices comprise at least one first conducting unit and at least one second conducting unit disposed outside the body.

In one embodiment, the environmental sensing device senses the current, voltage, capacitance, and temperature corresponding to the first conducting unit and the second conducting unit by the electric measuring unit, and a moisture of the surrounding environment is further calculated by the processing unit.

In one embodiment, the moisture provided by the disclosed environmental sensing device is more accurate because it is calculated according to the values of current, voltage, capacitance, and temperature combined with other environmental data.

As discussed above, in one embodiment, the only elements of the disclosed environmental sensing device that reside outside the environmental sensing device interior/accommodating space are the at least one first conducting unit and at least one second conducting unit.

As also discussed above, in one embodiment, the at least one first conducting unit and at least one second conducting unit are attached to, or are recessed below, the exterior surface of the environmental sensing device body such that the at least one first conducting unit and at least one second conducting unit extend no more than 3 millimeters from the exterior surface of the environmental sensing device body.

Consequently, in one embodiment, the disclosed environmental sensing devices have no probes or other features that extend more than 3 millimeters from the environmental sensing device body and environmental sensing device exterior surface.

In addition, in one embodiment, the disclosed environmental sensing device body is either spherical, cubic, ellipsoidal, or has a length dimension of the environmental sensing device body is no more than twice a width or diameter dimension of the environmental sensing device.

As a result, the disclosed environmental sensing devices are rugged, waterproof, and have no exterior features that extend more than 3 millimeters from the environmental sensing device body. Thus, in contrast to the prior art, the disclosed environmental sensing devices are extremely rugged and are designed to, and are capable of, being buried with the crops and stay with the crops during the entire lifetime of the crops.

Consequently, the disclosed environmental sensing devices and the methods for their use allow the disclosed environmental sensing devices to be buried with the crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops are they are transported, then remain with the crops as the crops loaded into storage units, and finally remain with the crops as the crops are stored; all using the same disclosed environmental sensing devices.

Consequently, as discussed in more detail below, the disclosed methods and systems provide a technical solution to the long-standing technical problem of accurately collecting data regarding the environmental factors and forces a given crop is subjected to at each stage of the lifetime of the crops using a single type of environmental sensing device.

### BRIEF DESCRIPTION OF THE DRAWINGS

FIG. 1A illustrates a perspective view of the environmental sensing device of a substantially cylindrical shape having an elliptical cross section in accordance with one embodiment.

FIG. 1B illustrates a perspective view of the environmental sensing device of a substantially cylindrical shape having a circular cross section in accordance with one embodiment.

FIG. 1C illustrates a perspective view of the environmental sensing device of a substantially cuboid shape in accordance with one embodiment.

FIG. 1D illustrates a perspective view of the environmental sensing device of a substantially spherical shape in accordance with one embodiment.

FIG. 1E illustrates a perspective view of the environmental sensing device of a substantially hexagonal shape in accordance with one embodiment.

FIG. 2 illustrates a functional block diagram of internal components of the environmental sensing device in accordance with one embodiment.

FIG. 3 illustrates a functional block diagram of internal components of the environmental sensing device in accordance with one embodiment.

FIG. 4A illustrates a cut away cross-sectional view of the environmental sensing device in accordance with one embodiment.

FIG. 4B illustrates a cut away cross-sectional view of the environmental sensing device and various components in accordance with one embodiment.

FIG. 4C illustrates a side cut away view of the environmental sensing device and various components in accordance with one embodiment.

FIG. 4D illustrates a top cut away view of the environmental sensing device and various components in accordance with one embodiment.

FIG. 4E illustrates a perspective diagonal cut away view of the environmental sensing device and various components in accordance with one embodiment.

FIG. 5 illustrates a schematic structural view of the first conducting unit and the second conducting unit in accordance with one embodiment.

FIG. 6 illustrates a flow chart of a process for measuring environmental conditions around crops according to the present invention.

FIG. 7 illustrates a flow chart showing steps of the application method of the environmental sensing device according to the present invention.

FIG. 8 illustrates a flow chart showing steps of the method for measuring environmental conditions around crops according to the present invention.

Common reference numerals are used throughout the figures and the detailed description to indicate like elements. One skilled in the art will readily recognize that the above figures are examples and that other architectures, modes of operation, orders of operation and elements/functions can be provided and implemented without departing from the characteristics and features of the invention, as set forth in the claims.

### DETAILED DESCRIPTION

Embodiments will now be discussed with reference to the accompanying figures (FIGs.), which depict one or more exemplary embodiments. Embodiments may be implemented in many different forms and should not be construed as limited to the embodiments set forth herein, shown in the FIGs., and/or described below. Rather, these exemplary embodiments are provided to allow a complete disclosure that conveys the principles of the invention, as set forth in the claims, to those of skill in the art.

The present invention is described by the following specific embodiments. Those with ordinary skills in the arts can readily understand other advantages and functions of the present invention after reading the disclosure of this specification. Any changes or adjustments made to their relative relationships, without modifying the substantial technical contents, are also to be construed as within the range implementable by the present invention.

Disclosed herein are various embodiments of a method and system that provide a technical solution to the long-standing technical problem of accurately collecting data regarding the environmental factors and forces a given crop is subjected to at each stage of the lifetime of the crops using a single type of environmental sensing device.

To this end, disclosed herein are methods and systems that provide a sensing device that can be buried with the crops and stay with the crops during the entire lifetime of the crops, i.e., a sensing device that can be buried with crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops as they are transported, then remain with the crops as the crops loaded into storage units, and finally remain with the crops as the crops are stored.

Consequently, the disclosed methods and systems provide a technical solution to the long-standing technical problem of accurately collecting data regarding the environmental factors and forces a given crop is subjected to at each stage of the lifetime of the crop using a single type of environmental sensing device.

In one embodiment, the disclosed environmental sensing device includes an environmental sensing device body that forms an environmental sensing device interior space, also referred to herein as an environmental sensing device accommodating space, and an environmental sensing device exterior surface. In one embodiment, the environmental sensing device interior/accommodating space is protected from the exterior environment by the environmental sensing device exterior surface such that the environmental sensing device interior/accommodating space is moisture resistant or moisture proof.

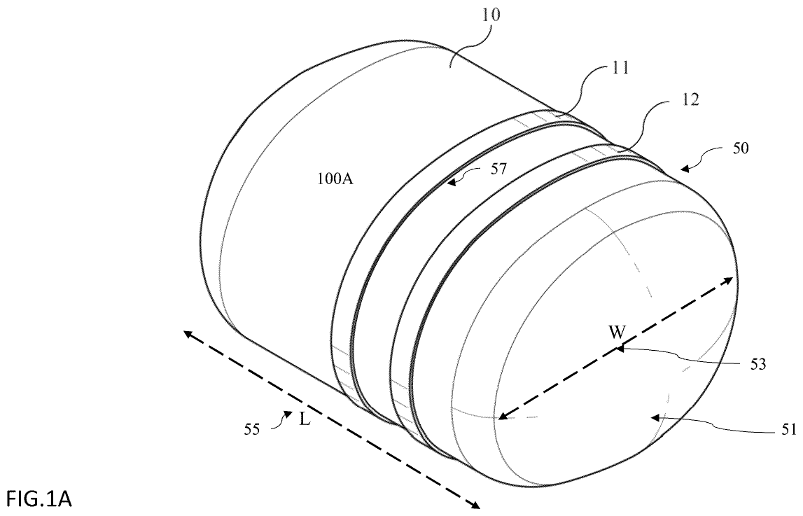

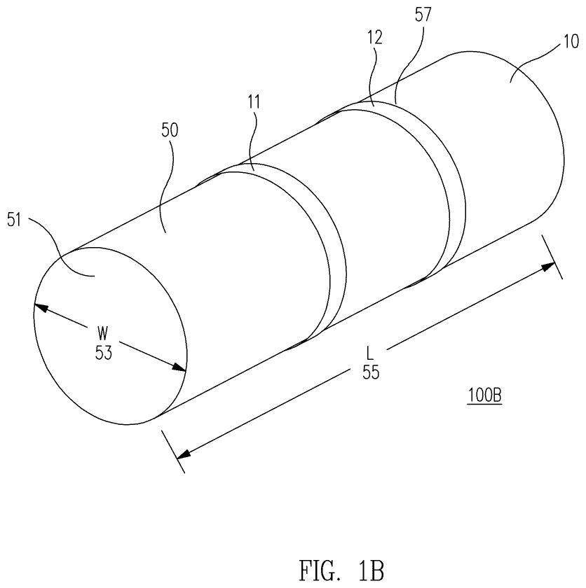

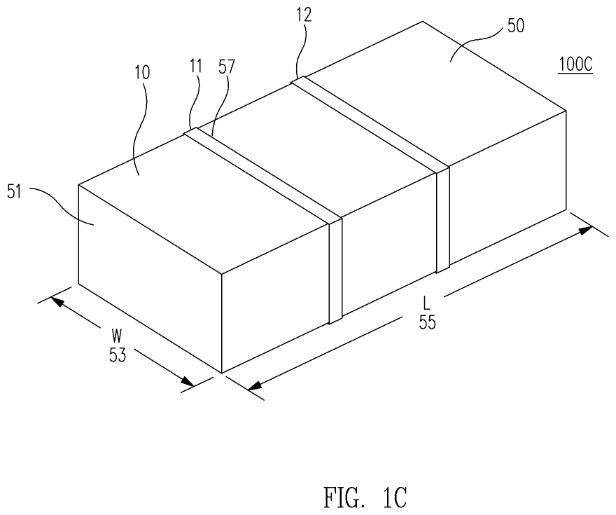

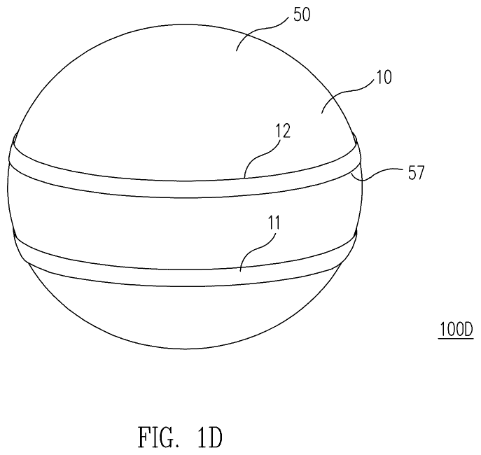

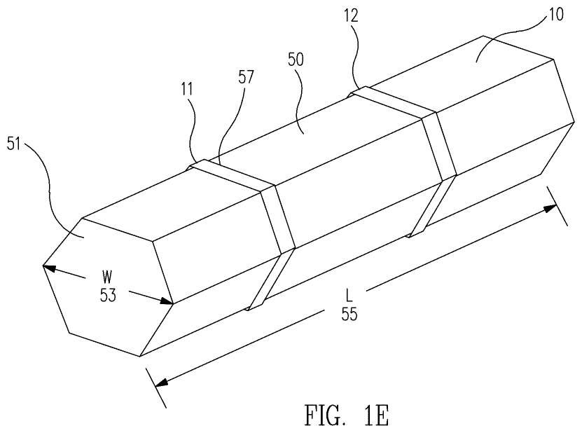

Referring to FIGS. 1A, 1B, 1C, 1D, 1E, 2 and 5 together, FIG. 1A illustrates a perspective view of an environmental sensing device 100A according to one embodiment. As seen in FIG. 1A, in one embodiment, environmental sensing device body 10 of environmental sensing device 100A, is formed in a substantially cylindrical shape with a substantially oval or elliptical cross section 51 wherein a length dimension “L” 55 of the environmental sensing device body 10 is no more than twice a width, or diameter, dimension “W” 53 of the environmental sensing device 100A.

FIG. 1B illustrates a perspective view of an environmental sensing device 100B according to one embodiment. As seen in FIG. 1B shows an environmental sensing device body 10 of an environmental sensing device 100B that is formed in a substantially cylindrical shape with a substantially circular cross section 51 wherein a length dimension “L” 55 of the environmental sensing device body 10 is no more than twice a width, or diameter, dimension “W” 53 of the environmental sensing device 100B.

FIG. 1C illustrates a perspective view of an environmental sensing device 100C according to one embodiment. As seen in FIG. 1C, in one embodiment, environmental sensing device body 10 of environmental sensing device 100C is formed in a substantially cuboid shape wherein a length dimension “L” 55 of the environmental sensing device body 10 is no more than twice a width dimension “W” 53 of the environmental sensing device 100C.

In other embodiments, environmental sensing device body 10 of environmental sensing device 100C is formed in a substantially cube shape (not shown).

FIG. 1D illustrates a perspective view of an environmental sensing device 100D according to one embodiment. As seen in FIG. 1D, in one embodiment, environmental sensing device body 10 of environmental sensing device 100D is formed in a substantially spherical shape.

FIG. 1E illustrates a perspective view of an environmental sensing device 100E according to one embodiment. As seen in FIG. 1E, in one embodiment, environmental sensing device body 10 of environmental sensing device 100E can be formed in a substantially multi-faced shape having a multi-side cross section 51 such as a triangular, hexagonal, octanal, etc., cross section wherein a length dimension “L” 55 of the environmental sensing device body 10 is no more than twice a width, or diameter, dimension “W” 53 of the environmental sensing device 100E.

In one embodiment, the environmental sensing device body is formed to have dimensions, e.g., length, width, girth, and volume similar to that of the crop with which it is be buried used (not shown).

Those of skill in the art will readily recognize that while five specific shapes of the disclosed environmental sensing device bodies 10 are shown in FIGS. 1A, 1B, 1C, 1D, and 1E for illustrative purposes, the disclosed environmental sensing devices can be formed to any shape, including various asymmetrical shapes, desired that can accommodate the electronics and features disclosed within the environmental sensing device body and that is study enough to withstand the forces imparted by mechanical harvesting, loading and unloading, transportation, and storage, as discussed herein, and/or as known in the art at the time of filing, and/or as developed made available after the time of filing.

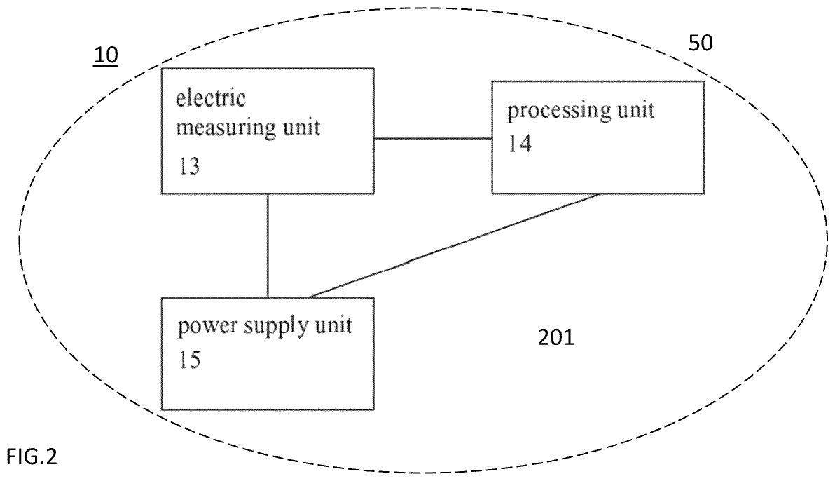

As discussed below, in one embodiment, environmental sensing device body 10 forms an environmental sensing device interior/accommodating space 201 (see FIGS. 2, 3, and 4) for placing various electronic components.

Since, in one embodiment, environmental sensing device body 10 is impact resistant, waterproof, and crash-resistant, the electronic components positioned within environmental sensing device body 10 are fully enclosed and protected from the outside environment and cannot be easily damaged. Therefore, the disclosed environmental sensing devices are capable of measuring environmental conditions more reliably and effectively than many prior art systems.

In one embodiment, environmental sensing device body 10 is formed to have dimensions and shape comparable to that of the crop to be monitored. For instance, in one embodiment, environmental sensing device 100A of FIG. 1A can be formed to have dimensions and shape comparable to that of a potato. Similarly, environmental sensing device 100D of FIG. 1D can be formed to have dimensions and shape comparable to that of a radish.

As noted above, those of skill in the art will readily recognize that while five specific shapes of the disclosed environmental sensing devices are shown in FIGS. 1A, 1B, 1C, 1D, and 1E for illustrative purposes, the disclosed environmental sensing devices can be formed to any shape desired, including various asymmetrical shapes, that can accommodate the electronics and features disclosed within the environmental sensing device body and that is study enough to withstand the forces imparted by mechanical harvesting, loading and unloading, transportation, and storage, as discussed herein, and/or as known in the art at the time of filing, and/or as developed made available after the time of filing.

In various embodiments, the environmental sensing device body 10 of FIGS. 1A through 1E can be formed of In various embodiments, environmental sensing device body 10 can be formed of Acrylonitrile Butadiene Styrene and polycarbonate (ABS+PC), High Density Polyethylene (HDPE). In some embodiments, the environmental sensing device body 10 can be formed using injection molding and PCBA. In other embodiments, environmental sensing device body 10 can be formed of any material discussed herein, and/or as available/known in the art at the time of filing, and/or as made available/becomes known after the time filing capable of being buried, harvested, loaded/unloaded, transported and stored with crops

As seen in FIGS. 1A through 1E, in one embodiment, the environmental sensing device body 10 includes at least one first conducting unit 11 and at least one second conducting unit 12 attached or formed into at least part of environmental sensing device exterior surface 50 of the environmental sensing device body 10.

In one embodiment, the at least one first conducting unit 11 and at least one second conducting unit 12 are attached to environmental sensing device exterior surface 50 of the environmental sensing device body 10 such that the at least one first conducting unit and at least one second conducting unit extend a distance 57 no more than 3 millimeters from the exterior surface 50 of the environmental sensing device body 10.

In one embodiment, the at least one first conducting unit 11 and at least one second conducting unit 12 are attached to environmental sensing device exterior surface 50 of the environmental sensing device body 10 such that the at least one first conducting unit and at least one second conducting unit extend a distance 57 no more than 2 millimeters from the exterior surface 50 of the environmental sensing device body 10.

In one embodiment, the at least one first conducting unit 11 and at least one second conducting unit 12 are attached to environmental sensing device exterior surface 50 of the environmental sensing device body 10 such that the at least one first conducting unit 11 and at least one second conducting unit 12 extend a distance 57 no more than 1 millimeter from the exterior surface 50 of the environmental sensing device body 10.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 do not extend beyond environmental sensing device exterior surface 50 of the environmental sensing device body 10 at all and are recessed in environmental sensing device exterior surface 50 of the environmental sensing device body 10.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 are recessed below environmental sensing device exterior surface 50 of the environmental sensing device body 10.

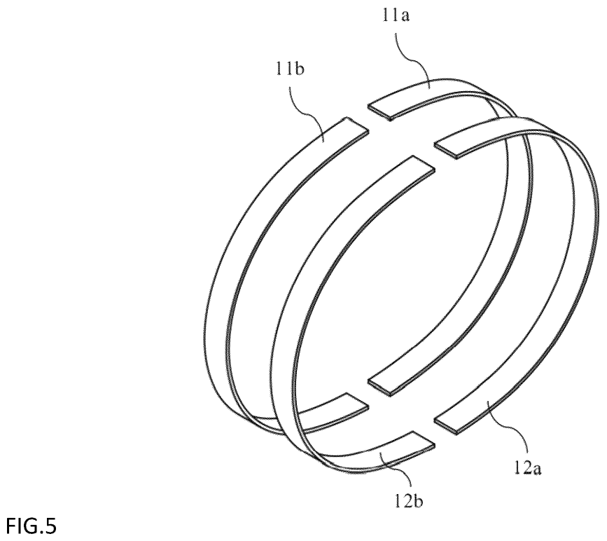

Referring to FIG. 5, as seen in FIG. 5, in one embodiment, the first conducting unit 11 is a two part annular first conducting unit 11a and 11b to be disposed around environmental sensing device exterior surface 50 of the environmental sensing device body 10 as shown in FIGS. 1A, 1B, 1C, 1D, and 1E.

As also shown in FIG. 5, in one embodiment, the second conducting unit 12 is a two part annular second conducting unit 12a and 12b to be disposed around environmental sensing device exterior surface 50 of the environmental sensing device body 10 as shown in FIGS. 1A, 1B, IC, 1D, and 1E.

FIG. 5 is discussed in more detail below.

In one embodiment, the first conducting unit 11 and the second conducting unit 12 are annular and disposed around environmental sensing device exterior surface 50 of the environmental sensing device body 10 in recessed regions formed in the exterior surface of the environmental sensing device body (not shown).

In one embodiment, the first conducting unit 11 and the second conducting unit 12 are made of stainless steel, titanium, plated gold, of any material discussed herein, and/or as available/known in the art at the time of filing, and/or as made available/becomes known after the time filing that is a conductor and corrosion resistant and is capable of being buried, harvested, loaded/unloaded, transported and stored with crops.

In one embodiment, the disclosed environmental sensing device includes an electric measuring unit, a processing unit, and a power supply unit. In one embodiment, the electric measuring unit, the processing unit, and the power supply unit are disposed entirely within the environmental sensing device interior/accommodating space 201 (see FIGS. 2, 3, and 4).

Referring to FIGS. 1A through 1E, 2, 3, 4, as noted above, in one embodiment, the disclosed environmental sensing devices of FIGS. 1A through 1E include an environmental sensing device body 10 that forms an environmental sensing device interior space, also referred to herein as an environmental sensing device accommodating space 201, and an environmental sensing device exterior surface 50. In one embodiment, the environmental sensing device interior/accommodating space 201 is protected from the exterior environment by the environmental sensing device exterior surface 50 such that the environmental sensing device interior/accommodating space is moisture proof.

FIG. 2 illustrates a functional block diagram of some of the internal components of the environmental sensing device according to one embodiment.

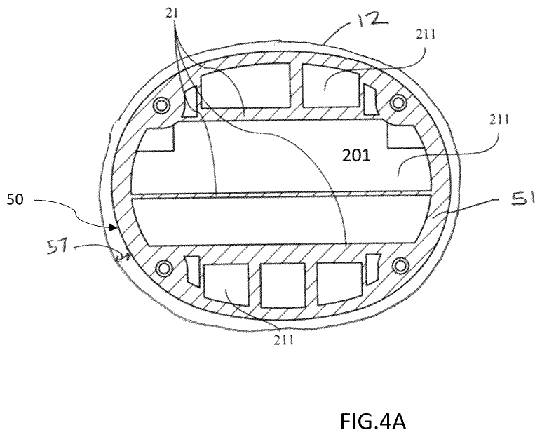

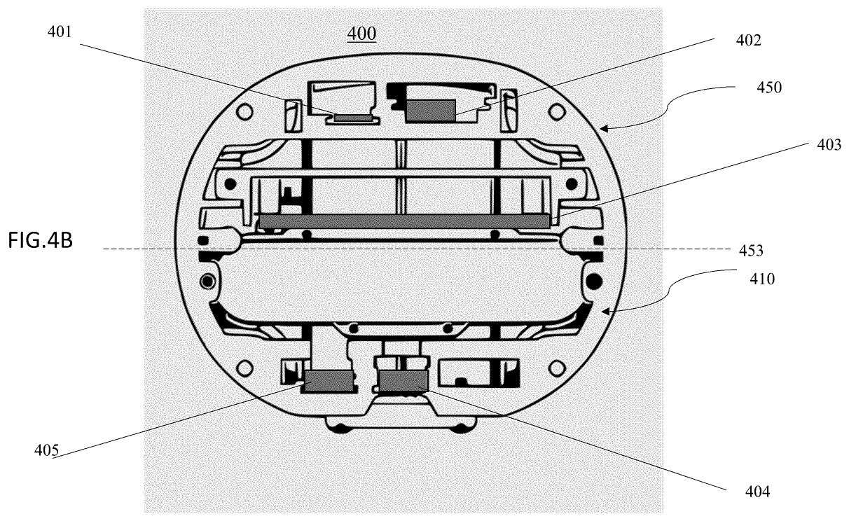

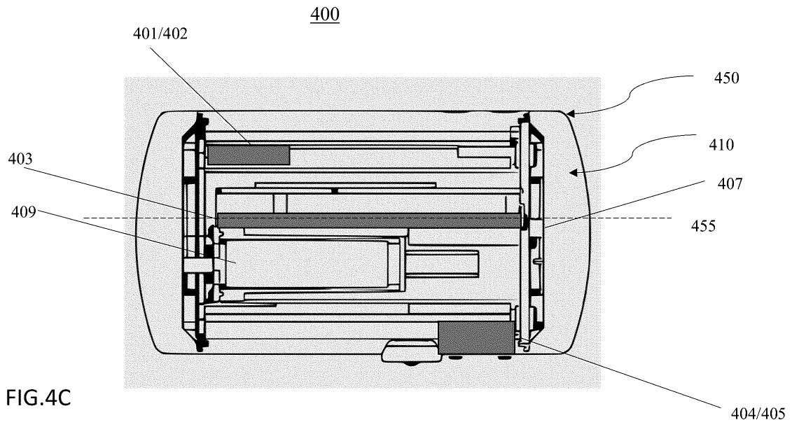

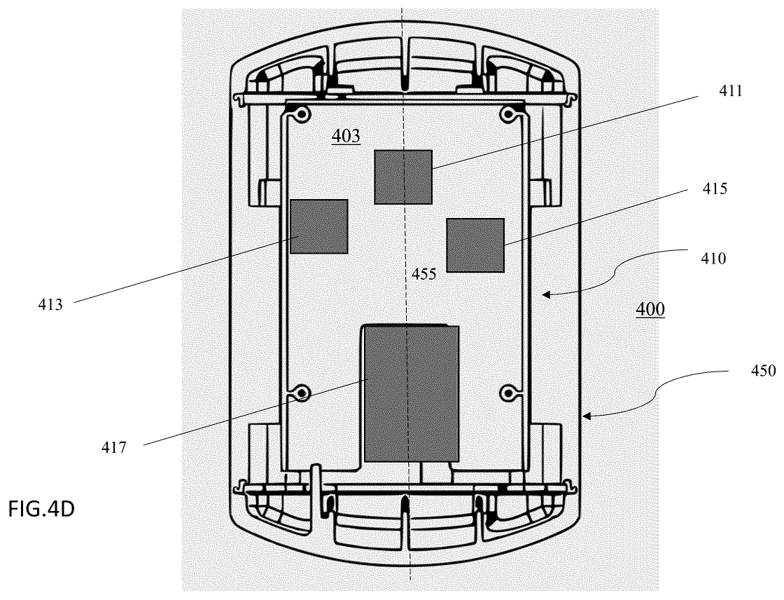

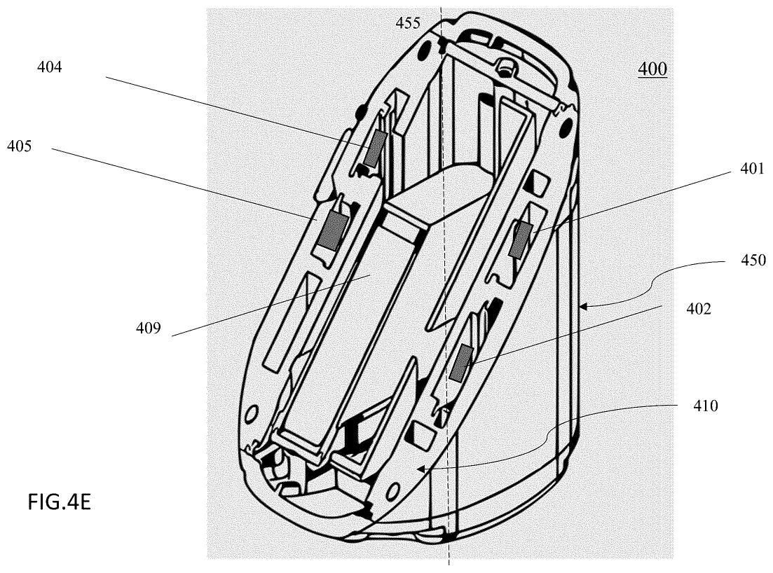

FIG. 4A show a cross sectional view of one embodiment of an environmental sensing device and such as environmental sensing devices 100A, 100B, 100C, 100D, and 100E.

Referring now FIGS. 1A through 1E, FIG. 2, FIG. 3, and FIG. 4A, together, as noted above, the environmental sensing devices 100A, 100B, 100C, 100D, and 100E include an environmental sensing device body 10, at least one first conducting unit 11, at least one second conducting unit 12. As seen in FIG. 2, an electric measuring unit 13, a processing unit 14, and a power supply unit 15 are also included and, in one embodiment, reside entirely within environmental sensing device interior/accommodating space 201 formed in environmental sensing device body 10.

Referring to FIG. 2, in one embodiment, electric measuring unit 13 measures current, voltage, capacitance, and temperature of the surrounding environment corresponding to the first conducting unit 11 and the second conducting unit 12.

In one embodiment, electric measuring unit 13 transmits a first signal including data indicating the measurement of the current, voltage, capacitance, and temperature of the environment.

In various embodiments, electric measuring unit 13 can be any electric measuring unit capable of collecting environmental data and transferring the data to processing unit 14, as discussed herein, and/or as known/available in the art at the time of filing, and/or as developed/made available after the time of filing.

In one embodiment, processing unit 14 is disposed entirely in the environmental sensing device interior/accommodating space 201 for receiving the first signal and calculating a moisture of the surrounding environment according to environmental data and the measurement of the current, voltage, capacitance, and temperature of the surrounding environment. In one embodiment, processing unit 14 is disposed in the environmental sensing device interior/accommodating space 201 and receives the first signal from electric measuring unit 13. In one embodiment, the processing unit 14 then processes the data to calculate the moisture of the surrounding environment according to environmental data and the measurements of current, voltage, capacitance, and temperature.

In one embodiment, the environmental data received by processing unit 14 includes the dielectric constant of soil or air at different moisture levels. In other words, the disclosed environmental sensing devices measure the moisture of the surrounding environment in real time. This can help manage the quality of the crops and also automate the process of doing so. For instance, the electric measuring unit 13 and the processing unit 14 may comprise, but are not limited to, an IC, a PLC or the like. In various other embodiments, processing unit 14 can be any processing unit capable of receiving data from electric measuring unit 13 and processing the data to calculate the moisture of the surrounding environment according to environmental data and the measurements of current, voltage, capacitance, and temperature, as discussed herein, and/or as known/available in the art at the time of filing, and/or as developed/made available after the time of filing.

In one embodiment, power supply unit 15 is disposed entirely in the environmental sensing device interior/accommodating space 201 for supplying power to electric measuring unit 13 and processing unit 14. In one embodiment, power supply unit 15 can be, for example, a battery or any suitable power supply. In various other embodiments, power supply unit 15 can be any power supply capable of providing power to electric measuring unit 13 and processing unit 14, as discussed herein, and/or as known/available in the art at the time of filing, and/or as developed/made available after the time of filing.

In various embodiments, in addition to electric measuring unit 13, processing unit 14, and power supply unit 15, the disclosed environmental sensing devices can also include other units disposed within environmental sensing device interior/accommodating space 201 for collecting and/or processing other types of data.

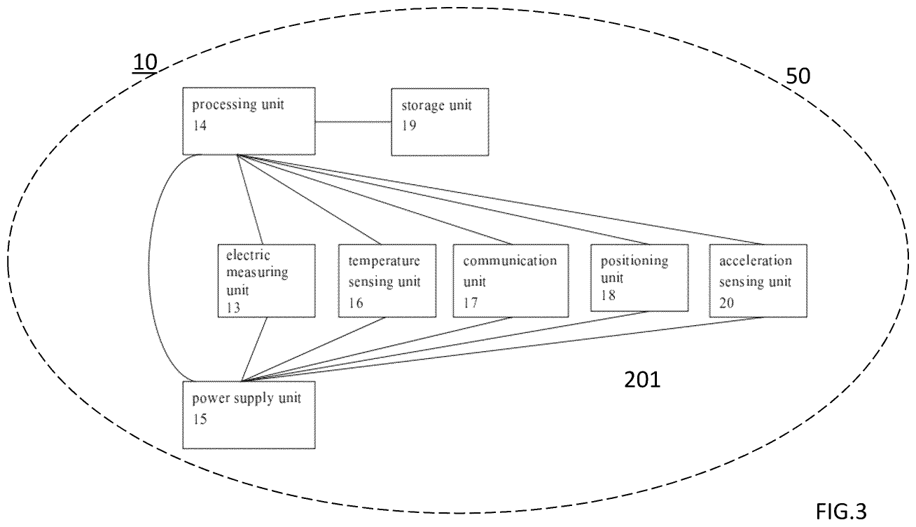

FIG. 3 shows a functional block diagram of the internal components of one embodiment of the disclosed environmental sensing devices.

Referring to FIGS. 1A, 1B, 1C, 1D, 1E, 3, and 4, as seen in FIG. 3, in one embodiment, the disclosed environmental sensing devices include a temperature sensing unit 16 disposed in the environmental sensing device interior/accommodating space 201 for sensing temperature of the surrounding environment.

In one embodiment, temperature sensing unit 16 transmits a second signal containing temperature data. In one embodiment, processing unit 14 receives the second signal and uses the second signal data to calibrate the measurement of the temperature of the surrounding environment according to the second signal, so as to obtain a more accurate temperature reading.

In one embodiment, the processing unit 14 calculates the moisture of the surrounding environment according to the environmental data, the measurement of the current, voltage, and capacitance of the surrounding environment and/or the calibrated temperature of the surrounding environment, etc. Because the capacitance is temperature dependent and moisture is affected by the temperature. The disclosed environmental sensing device can provide a more accurate and precise temperature reading of the surrounding environment by the calibration of the temperature sensing unit 16, resulting in a more reliable calculation of moisture than prior art systems can provide.

In various embodiments, temperature sensing unit 16 can be any temperature sensing unit capable of sensing temperature of the surrounding environment and transmitting a second signal as discussed herein, and/or as known/available in the art at the time of filing, and/or as developed/made available after the time of filing.

In one embodiment, the disclosed environmental sensing devices include a communication unit 17 disposed in the environmental sensing device interior/accommodating space and electrically connected to the processing unit for communicating with an external data processing device, such as a cellphone or personal computer.

In one embodiment, communication unit 17, including a data transmission device such as an antenna, is entirely disposed within the environmental sensing device interior/accommodating space.

In one embodiment, communication unit 17 is electrically connected to processing unit 14 for wirelessly communicating with an external data processing device.

In one embodiment, communication unit 17 may be a wireless communication component disposed within environmental sensing device interior/accommodating space 201 using LTE technology (CAT-M1, NB-IoT), but it is not limited thereto.

In various embodiments, communication unit 17 can be a WiFi communication unit, a LoRa communication unit, a Cellular communication unit, a Bluetooth communication unit, and/or any communication unit as discussed herein, and/or as available/known in the art at the time of filing, and/or as made available/becomes known after the time filing that does not require an antenna that extends more than 3 mm from the environmental sensing device body.

In one embodiment, the disclosed environmental sensing devices exchange information with an external data processing device (such as a mobile device, a computer, a server, etc.) through communication unit 17.

In one embodiment, the disclosed environmental sensing devices transmit real-time information such as moisture levels and temperature to the external data processing device or receive environmental data from the external data processing device wirelessly using an internal data transmission device disposed entirely within environmental sensing device interior/accommodating space 201.

In various embodiments, communication unit 17 can be any communication unit 17 capable communicating with an external data processing device, such as a cellphone or personal computer, using an internal antenna/data transmission device housed entirely within environmental sensing device interior/accommodating space 201, as discussed herein, and/or as known/available in the art at the time of filing, and/or as developed/made available after the time of filing.

In one embodiment, the disclosed environmental sensing devices also include a positioning unit 18 disposed in the environmental sensing device interior/accommodating space 201 and electrically connected to processing unit 14 for tracking the location of the environmental sensing device, and therefore the crops with which the environmental sensing device resides, during the entire growth and processing cycles.

In one embodiment, positioning unit 18 is an electronic component such as a global positioning system (GPS) module, but it is not limited thereto. In one embodiment, the disclosed environmental sensing devices track the location of the environmental sensing device using the positioning unit 18 which can be useful in identifying conditions of the crops, such as whether the crops are currently at the stage of growing, transportation or storage, etc.

In one embodiment, communication unit 17 transmits a third signal indicating the location of the environmental sensing device to an external database (not shown). In one embodiment, the database includes pre-saved worldwide geographic information or meteorological information.

In one embodiment, the database sends the geographic information or meteorological information of the location of the environmental sensing device to communication unit 17 based on the data included in the third signal.

In one embodiment, processing unit 14 then calculates the moisture of the surrounding environment according to the environmental data, such as the measurement of the current, voltage, and capacitance of the surrounding environment and the calibrated temperature of the surrounding environment and the geographic information or meteorological information, etc.

Since soil moisture reacts differently in different soil types, the disclosed environmental sensing device can be pre-calibrated so as to correspond to different soil types, such as sand, silt, clay, etc. In one embodiment, the positioning unit 18 is used to determine the type of soil in which the environmental sensing device is disposed, by communicating with the database and the environmental sensing device can then automatically switch the soil moisture profile to match that of the soil in which the environmental sensing device, and therefore the crop, is currently disposed, so as to provide a more accurate moisture reading.

In various embodiments, positioning unit 18 can be any positioning unit capable of tracking the location of the environmental sensing device, as discussed herein, and/or as known/available in the art at the time of filing, and/or as developed/made available after the time of filing.

In one embodiment, the disclosed environmental sensing devices include a storage unit 19 disposed in the environmental sensing device interior/accommodating space 201 for receiving and recording the measurement of the current, voltage, capacitance, and temperature of the surrounding environment, the moisture calculated by the processing unit and the corresponding time.

In one embodiment, storage unit 19 receives and records the measurement of the current, voltage, capacitance, and temperature of the surrounding environment, the data of the moisture calculated by the processing unit 14, and the relative event/time log, etc. In one embodiment, storage unit 19 can be a memory, but it is not limited thereto. Environmental data such as changes of moisture through a predetermined period of time, can be stored and accessed in the storage unit 19. Further, the storage unit 19 may be used as a back-up memory to save the measurements and/or the calculated values by the disclosed environmental sensing device.

In various embodiments, storage unit 19 can be any storage unit 19 capable of receiving and recording the measurement of the current, voltage, capacitance, and temperature of the surrounding environment, the moisture calculated by the processing unit and the corresponding time, as discussed herein, and/or as known/available in the art at the time of filing, and/or as developed/made available after the time of filing.

In one embodiment, the disclosed environmental sensing device further includes an acceleration sensing unit 20 disposed in the environmental sensing device interior/accommodating space 201 and electrically connected to processing unit 14 for sensing an external force received by/imposed on environmental sensing device body 10.

In one embodiment, acceleration sensing unit 20 can be a 3-axis accelerometer, but it is not limited thereto. In one embodiment, since the disclosed environmental sensing devices are designed to remain with the crops at all stages of the crop processing cycle, including during harvesting, loading, transporting, unloading and storing acceleration, sensing unit 20 can sense the external forces received by/imposed on the crops during the entire crop processing cycle.

For instance, in one embodiment, the external forces may be incurred by other objects hitting the environmental sensing device or by the environmental sensing device hitting something else. Crops may be harvested using heavy machinery and pass through multiple pieces of machinery and locations, and as such there is a high potential for them to be dropped from excessive heights or at excessive speeds which can cause bruising to the crops. Bruising may reduce the quality and quantity of the crops and may further in turn create disease. This process can be monitored by the disclosed environmental sensing device, this data can then be used advise the farmers on how to better manage their harvesting and crop transportation operations. In other instances the data can be used to train ML and/or AI models, and update these models, to provide predictive data and again advise the farmers on how to better manage their harvesting and crop transportation operations. According to one embodiment, the environmental sensing device is rugged, waterproof, and has no external components extending more than 3 millimeters, to allow the users to harvest the crops without having to remove the disclosed environmental sensing device from the field.

In one embodiment, acceleration sensing unit 20 can be any acceleration sensing unit capable of sensing an external force received by/imposed on environmental sensing device body 10, as discussed herein, and/or as known/available in the art at the time of filing, and/or as developed/made available after the time of filing.

As noted above, FIG. 4A shows a specific illustrative example of a cross-sectional view of one embodiment of the disclosed environmental sensing devices.

Referring to FIG. 4A, in one embodiment, the disclosed environmental sensing devices can include a fixing frame 21 disposed in the environmental sensing device interior/accommodating space 201 for securing components disposed in the environmental sensing device interior/accommodating space 201. The fixing frame 21 may divide the environmental sensing device interior/accommodating space 201 into a one or more sub-cavities 211. Consequently, in one embodiment, fixing frame 21 may have one or more sub-cavities 211 illustrated in FIG. 4A. The components of the environmental sensing device according to the present invention may then be fixed and secured in the cavities correspondingly.

In one embodiment, the disclosed environmental sensing devices may comprise one or more internal antennas (not shown) which may slide into their own independent cavities (not shown). These cavities for the antennas may be designed to be as far away from battery/power supply unit 15 as possible so as to avoid interference, in their own cavities to protect them and located there because that place may promote the most optimal signal strength.

FIGS. 4B, 4C, 4D, and 4E show various views of specific component placements in various one or more sub-cavities 211 of FIG. 4A, in accordance with one specific illustrative example of one embodiment.

In particular, FIG. 4B illustrates a cut away cross-sectional view of a disclosed environmental sensing device 400, such as environmental sensing devices 100A, 100B, 100C, 100D, and 100E, and various components in accordance with one embodiment.

FIG. 4C illustrates a side cut away view of the disclosed environmental sensing device 400 and various components in accordance with one embodiment.

FIG. 4D illustrates a top cut away view of the disclosed environmental sensing device 400 and various components in accordance with one embodiment.

FIG. 4E illustrates a perspective diagonal cut away view of the disclosed environmental sensing device 400 and various components in accordance with one embodiment.

Referring now to FIGS. 4A, 4B, 4C, 4D and 4E together, as shown in FIGS. 4B, 4C, 4D and 4E, in one embodiment, environmental sensing device 400 includes environmental sensing device body 410 and environmental sensing device exterior surface 450. In addition, environmental sensing device 400 includes longitudinal length axis 455 and horizontal width axis 453.

As also shown in FIGS. 4B, 4C, 4D and 4E, in one embodiment, environmental sensing device 400 includes LTE antenna 401, GPS antenna 402, main processing unit board 403, ambient temperature and/or humidity sensor 404, moisture sensor 405, charging port 407, and battery power supply 409, each positioned within a respective sub-cavity of sub-cavities 211 of FIG. 4A.

In addition, FIG. 4D shows processing unit board 403 including Micro Controller Unit 411, Bluetooth Low Energy 413, Inertial Measurement Unit 415, and modem 417.

As a result of the structure and features shown in FIGS. 4A, 4B, 4C, 4D, and 4E, including the placement of the various components in respective sub-cavities 211 of FIG. 4A, moving or shaking the disclosed environmental sensing devices, such during harvesting, loading, unloading and transportation, will not cause damage to the internal components or reduce the efficacy of the disclosed environmental sensing devices. In other words, fixing frame 21 may protect the components from undesired conditions such as vibrations and drops that the environmental sensing device may incur to ensure continuous operation of the environmental sensing device.

The features of FIGS. 4A, 4B, 4C, 4D, and 4E combined with the fact that, as discussed above, in one embodiment, the only elements of the disclosed environmental sensing device that reside outside the environmental sensing device interior/accommodating space 201 are the at least one first conducting unit 11 and at least one second conducting unit 12 make the disclosed environmental sensing devices extremely sturdy and well suited for remaining with the crops at every stage of the growth and processing cycles.

As also discussed above, in one embodiment, the at least one first conducting unit 11 and at least one second conducting unit 11 are attached to, or recessed in, the exterior surface of the environmental sensing device body such that the at least one first conducting unit and at least one second conducting unit extend no more than 3 millimeters from the exterior surface of the environmental sensing device body.

Consequently, in one embodiment, the disclosed environmental sensing devices have no probes or other features that extend more than 3 millimeters from the environmental sensing device body and environmental sensing device exterior surface.

In addition, in one embodiment, the disclosed environmental sensing device body is either cylindrical, cuboid, spherical, or otherwise has a length dimension of the environmental sensing device body that is no more than twice a width or diameter dimension of the environmental sensing device.

Therefore, according to the disclosed embodiments, a fixing frame 21 increases reliability of the disclosed environmental sensing devices, the disclosed environmental sensing devices have no probes or other features that extend more than 3 millimeters from the environmental sensing device body and environmental sensing device exterior surface, and the disclosed environmental sensing device body is either cylindrical, spherical, cubic, and has a length dimension that is no more than twice a width or diameter dimension of the environmental sensing device. In addition, in one embodiment, environmental sensing device body 10 of the disclosed environmental sensing devices may include a plastic cover or cushion for the components to act as a further shield during any drops or impacts.

Thus, in contrast to the prior art, the combination of these and other features allow the disclosed environmental sensing devices to be buried with the crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops are they are transported, then remain with the crops as the crops loaded into storage units, and finally remain with the crops as the crops are stored; all using a single disclosed environmental sensing device.

As noted above, in one embodiment, the at least one first conducting unit 11 and at least one second conducting unit 12 are attached to environmental sensing device exterior surface 50 of the environmental sensing device body 10 such that the at least one first conducting unit and at least one second conducting unit extend no more than 3 millimeters from the exterior surface of the environmental sensing device body.

In one embodiment, the at least one first conducting unit 11 and at least one second conducting unit 12 are attached to environmental sensing device exterior surface 50 of the environmental sensing device body 10 such that the at least one first conducting unit and at least one second conducting unit extend no more than 2 millimeters from the exterior surface of the environmental sensing device body.

In one embodiment, the at least one first conducting unit 11 and at least one second conducting unit 12 are attached to environmental sensing device exterior surface 50 of the environmental sensing device body 10 such that the at least one first conducting unit 11 and at least one second conducting unit 12 extend no more than 1 millimeter from the exterior surface of the environmental sensing device body.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 do not extend outside environmental sensing device exterior surface 50 of the environmental sensing device body 10 are substantially flush with environmental sensing device exterior surface 50 of the environmental sensing device body 10.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 are recessed below environmental sensing device exterior surface 50 of the environmental sensing device body 10.

FIG. 5 illustrates a schematic structural view of one illustrative example of one embodiment of the first conducting unit 11 and the second conducting unit 12.

As shown in FIG. 5, in one embodiment, the first conducting unit 11 is a two part annular first conducting unit including first conducting unit portions 11a and 11b to be disposed around environmental sensing device exterior surface 50 of the environmental sensing device body 10 as shown in FIGS. 1A, 1B, 1C, 1D, and 1E.

Similarly, as also shown in FIG. 5, in one embodiment, the second conducting unit 12 is a two part annular second conducting unit including second conducting unit portions 12a and 12b to be disposed around environmental sensing device exterior surface 50 of the environmental sensing device body 10 as shown in FIGS. 1A, 1B, 1C, 1D, and 1E.

Referring now to FIGS. 1A, 1B, 1C, 1D, 1E, 3, 4, and 5 together, in one embodiment, first conducting unit portions 11a, 11b and the second conducting portions 12a, 12b are designed in a shape of an arc or an arch, or any other shape and/or of any number or portions, in accordance with the shape of the body 10.

In one embodiment, the first conducting unit portions 11a, 11b and the second conducting unit portions 12a, 12b can be disposed around the outside of the body 10. It should be noted that, in the embodiment of FIG. 5, there are a set of two first conducting unit portions 11a, 11b and a set of two second conducting unit portions 12a, 12b. In this embodiment, the electric measuring unit 13 can sense measurements (including the current, the voltage, the capacitance and the temperature) of the two sets of the first conducting unit portions 11a, 11b and the second conducting unit portions 12a, 12b.

By these two sets of measurements, two measures of environmental moisture can be calculated by the environmental sensing device of the present invention; one is the environmental moisture corresponding to the positions of the first conducting unit portion 11a and the second conducting unit portion 12a, and the other is the environmental moisture corresponding to the positions of the first conducting unit portion 11b and the second conducting unit portion 12b.

In various embodiments, the number of the first conducting unit portions and the second conducting unit portions can be increased or decreased in sets as needed to sense the environmental moisture, as more moisture readings may provide a more accurate measurement of the environmental conditions.

In another embodiment (not shown), the disclosed environmental sensing device may comprise at least two first conducting units 11, at least two second conducting units 12 and at least two electric measuring units (not shown), such as electric measuring unit 13. Each of the electric measuring units may measure the current, the voltage, the capacitance and the temperature, etc., of each set of the first conducting units 11 and the second conducting units 12.

In another embodiment (not shown), the disclosed environmental sensing device may comprise two or more temperature sensing units, such as temperature sensing unit 16.

Regarding the abovementioned embodiments, the at least two conducting units and electric measuring units and the at least two temperature sensing units may be functionally independent (i.e. the conducting units, the electric measuring units and the temperature sensing units have no overlapping functions to each other) to better improve the accuracy and to prevent any data inconsistencies. The conducting units, the electric measuring units and the temperature sensing units are also omitted from accessing history data to prevent any discrepancies which might influence the correctness of data.

According to another embodiment (not shown), the environmental sensing device may further comprise a common moisture sensor which is functionally independent of the environmental sensing device. The common moisture sensor may be any moisture sensors known in the art. In this embodiment, the common moisture sensor may sense the moisture, which may be compared with the moisture calculated by the processing unit 14 for the users' reference.

In one embodiment, the first conducting unit 11 and the second conducting unit 12 can be made of conduction materials which are resistant to oxidization, such as stainless steel or the like, but they are not limited thereto. Since easily oxidized materials such as copper or copper plating material may degrade device performance or contaminate the crop and the soil.

As noted, in one embodiment, the first conducting unit 11 and the second conducting unit 12 may be made of stainless steel. It also ensures a longer life of the environmental sensing device of the present invention.

In one embodiment, the disclosed environmental sensing device may further conduct the various calculations with a noise filtering algorithm. For power consumption reasons, signals emitted by the first conducting unit and the second conducting unit may be low powered. In order to reduce the impact of background noise and unpredictable environmental surroundings, several techniques may be used in the present invention.

For example, to eliminate white noise type interference like thermal noise, the same signal may be sent multiple times and a low pass filter may be used. Environmental interferences are more unpredictable, and due to an object may responses differently on different frequency, multiple different frequencies are used and the results are compared to get a coherent trend. After the two stages, the number is kept in a storage buffer. Then filters, such as Kalman filter or the like, may be used to deprive the longer trend.

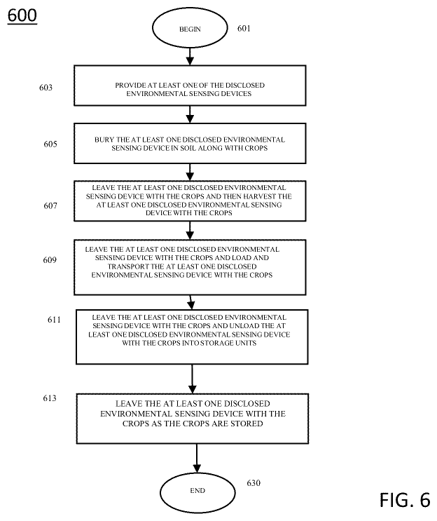

In some embodiments, a method of monitoring crops is disclosed. FIGS. 6, 7 and 8 show various embodiments of disclosed methods and processes for monitoring crops in accordance with the invention.

Referring to FIG. 6, FIG. 6 is a flow chart of a process 600 for monitoring crops in accordance with one embodiment.

As seen in FIG. 6, process 600 begins at 601 and process flow proceeds to operation 603. In one embodiment, at operation 603, at least one of the disclosed environmental sensing devices discussed above with respect to FIGS. 1, 1B, 1C, 1D, 1E, 2, 3, 4, and 5 is provided.

Referring to FIGS. 1, 1B, 1C, 1D, 1E, 2, 3, 4, 5, and 6 together in one embodiment, the at least one disclosed environmental sensing device provided at 603 can be buried with the crops and stay with the crops during the entire lifetime of the crops, i.e., a sensing device that can be buried with crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops are they are transported, then remain with the crops as the crops loaded into storage units, and finally remain with the crops as the crops are stored.

Referring to FIGS. 1A, 1B, 1C, 1D, 1E, 2 and 5 together, FIG. 1A illustrates a perspective view of an environmental sensing device 100A provided at operation 603 according to one embodiment. As seen in FIG. 1A, in one embodiment, environmental sensing device body 10 of environmental sensing device 100A, is formed in a substantially cylindrical shape with a substantially oval or elliptical cross section 51 wherein a length dimension “L” 55 of the environmental sensing device body 10 is no more than twice a width, or diameter, dimension “W” 53 of the environmental sensing device 100A.

FIG. 1B illustrates a perspective view of an environmental sensing device 100B provided at operation 603 according to one embodiment. As seen in FIG. 1B shows an environmental sensing device body 10 of an environmental sensing device 100B that is formed in a substantially cylindrical shape with a substantially circular cross section 51 wherein a length dimension “L” 55 of the environmental sensing device body 10 is no more than twice a width, or diameter, dimension “W” 53 of the environmental sensing device 100B.

FIG. 1C illustrates a perspective view of an environmental sensing device 100C provided at operation 603 according to one embodiment. As seen in FIG. 1C, in one embodiment, environmental sensing device body 10 of environmental sensing device 100C is formed in a substantially cuboid shape wherein a length dimension “L” 55 of the environmental sensing device body 10 is no more than twice a width dimension “W” 53 of the environmental sensing device 100C.

In other embodiments, environmental sensing device body 10 of environmental sensing device 100C provided at operation 603 is formed in a substantially cube shape (not shown).

FIG. 1D illustrates a perspective view of an environmental sensing device 100D provided at operation 603 according to one embodiment. As seen in FIG. 1D, in one embodiment, environmental sensing device body 10 of environmental sensing device 100D is formed in a substantially spherical shape.

FIG. 1E illustrates a perspective view of an environmental sensing device 100E provided at operation 603 according to one embodiment. As seen in FIG. 1E, in one embodiment, environmental sensing device body 10 of environmental sensing device 100E can be formed in a substantially multi-faced shape having a multi-side cross section 51 such as a triangular, hexagonal, octanal, etc., cross section wherein a length dimension “L” 55 of the environmental sensing device body 10 is no more than twice a width, or diameter, dimension “W” 53 of the environmental sensing device 100E.

In one embodiment, the environmental sensing device body is formed to have dimensions, e.g., length, width, girth, and volume similar to that of the crop with which it is be buried used (not shown).

Those of skill in the art will readily recognize that while five specific shapes of the disclosed environmental sensing device bodies 10 are shown in FIGS. 1A, 1B, 1C, 1D, and 1E for illustrative purposes, the disclosed environmental sensing devices can be formed to any shape, including various asymmetrical shapes, desired that can accommodate the electronics and features disclosed within the environmental sensing device body and that is study enough to withstand the forces imparted by mechanical harvesting, loading and unloading, transportation, and storage, as discussed herein, and/or as known in the art at the time of filing, and/or as developed made available after the time of filing.

In one embodiment, environmental sensing device body 10 is waterproof and impact resistant. In various embodiments, the environmental sensing device body can be formed of Acrylonitrile Butadiene Styrene and polycarbonate (ABS+PC), High Density Polyethylene (HDPE). In some embodiments, the environmental sensing device body can be formed using injection molding and PCBA. In other embodiments, the environmental sensing device body can be formed of any material discussed herein, and/or as available/known in the art at the time of filing, and/or as made available/becomes known after the time filing capable of being buried, harvested, loaded/unloaded, transported and stored with crops.

As discussed below, in one embodiment, environmental sensing device body 10 forms an environmental sensing device interior/accommodating space 201 (see FIGS. 2, 3, and 4) for placing various electronic components.

Since, in one embodiment, environmental sensing device body 10 is impact resistant, waterproof, and crash-resistant, the electronic components positioned within environmental sensing device body 10 are fully enclosed and protected from the outside environment and cannot be easily damaged. Therefore, the disclosed environmental sensing devices are capable of measuring environmental conditions more reliably and effectively than many prior art systems.

In one embodiment, environmental sensing device body 10 is formed to have dimensions and shape comparable to that of the crop to be monitored. For instance, in one embodiment, environmental sensing device 100A of FIG. 1A can be formed to have dimensions and shape comparable to that of a potato. Similarly, environmental sensing device 100D of FIG. 1D can be formed to have dimensions and shape comparable to that of a radish.

As noted above, those of skill in the art will readily recognize that while five specific shapes of the disclosed environmental sensing devices are shown in FIGS. 1A, 1B, 1C, 1D, and 1E for illustrative purposes, the disclosed environmental sensing devices can be formed to any shape desired, including various asymmetrical shapes, that can accommodate the electronics and features disclosed within the environmental sensing device body and that is study enough to withstand the forces imparted by mechanical harvesting, loading and unloading, transportation, and storage, as discussed herein, and/or as known in the art at the time of filing, and/or as developed made available after the time of filing.

In various embodiments, the environmental sensing device body 10 of FIGS. 1A through 1E can be formed of Acrylonitrile Butadiene Styrene and polycarbonate (ABS+PC), High Density Polyethylene (HDPE). In some embodiments, the environmental sensing device body can be formed using injection molding and PCBA. In other embodiments, the environmental sensing device body can be formed of any material discussed herein, and/or as available/known in the art at the time of filing, and/or as made available/becomes known after the time filing capable of being buried, harvested, loaded/unloaded, transported and stored with crops

As seen in FIGS. 1A through 1E, in one embodiment, the environmental sensing device body 10 includes at least one first conducting unit 11 and at least one second conducting unit 12 attached or formed into at least part of environmental sensing device exterior surface 50 of the environmental sensing device body 10.

In one embodiment, the at least one first conducting unit 11 and at least one second conducting unit 12 are attached to environmental sensing device exterior surface 50 of the environmental sensing device body 10 such that the at least one first conducting unit and at least one second conducting unit extend a distance 57 no more than 3 millimeters from the exterior surface 50 of the environmental sensing device body 10.

In one embodiment, the at least one first conducting unit 11 and at least one second conducting unit 12 are attached to environmental sensing device exterior surface 50 of the environmental sensing device body 10 such that the at least one first conducting unit and at least one second conducting unit extend a distance 57 no more than 2 millimeters from the exterior surface 50 of the environmental sensing device body 10.

In one embodiment, the at least one first conducting unit 11 and at least one second conducting unit 12 are attached to environmental sensing device exterior surface 50 of the environmental sensing device body 10 such that the at least one first conducting unit 11 and at least one second conducting unit 12 extend a distance 57 no more than 1 millimeter from the exterior surface 50 of the environmental sensing device body 10.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 do not extend beyond environmental sensing device exterior surface 50 of the environmental sensing device body 10 at all and are recessed in environmental sensing device exterior surface 50 of the environmental sensing device body 10.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 are recessed below environmental sensing device exterior surface 50 of the environmental sensing device body 10.

Referring to FIG. 5, as seen in FIG. 5, in one embodiment, the first conducting unit 11 is a two part annular first conducting unit 11a and 11b to be disposed around environmental sensing device exterior surface 50 of the environmental sensing device body 10 as shown in FIGS. 1A, 1B, 1C, 1D, and 1E.

As also shown in FIG. 5, in one embodiment, the second conducting unit 12 is a two part annular second conducting unit 12a and 12b to be disposed around environmental sensing device exterior surface 50 of the environmental sensing device body 10 as shown in FIGS. 1A, 1B, 1C, 1D, and 1E.

FIG. 5 is discussed in more detail below.

In one embodiment, the first conducting unit 11 and the second conducting unit 12 are annular and disposed around environmental sensing device exterior surface 50 of the environmental sensing device body 10 in recessed regions formed in the exterior surface of the environmental sensing device body (not shown).

In one embodiment, the first conducting unit 11 and the second conducting unit 12 are made of stainless steel, titanium, plated gold, of any material discussed herein, and/or as available/known in the art at the time of filing, and/or as made available/becomes known after the time filing that is a conductor and corrosion resistant and is capable of being buried, harvested, loaded/unloaded, transported and stored with crops.

In one embodiment, the disclosed environmental sensing device includes an electric measuring unit, a processing unit, and a power supply unit. In one embodiment, the electric measuring unit, the processing unit, and the power supply unit are disposed entirely within the environmental sensing device interior/accommodating space 201 (see FIGS. 2, 3, and 4).

Referring to FIGS. 1A through 1E, 2, 3, 4, as noted above, in one embodiment, the disclosed environmental sensing devices of FIGS. 1A through 1E include an environmental sensing device body 10 that forms an environmental sensing device interior space, also referred to herein as an environmental sensing device accommodating space 201, and an environmental sensing device exterior surface 50. In one embodiment, the environmental sensing device interior/accommodating space 201 is protected from the exterior environment by the environmental sensing device exterior surface 50 such that the environmental sensing device interior/accommodating space is moisture proof.

FIG. 2 illustrates a functional block diagram of some of the internal components of the environmental sensing device according to one embodiment.

FIG. 4A show a cross sectional view of one embodiment of an environmental sensing device such as environmental sensing devices 100A, 100B, 100C, 100D, and 100E.

Referring now FIGS. 1A through 1E, FIG. 2, FIG. 3, and FIG. 4A together, as noted above, the environmental sensing devices 100A, 100B, 100C, 100D, and 100E include an environmental sensing device body 10, at least one first conducting unit 11, at least one second conducting unit 12. As seen in FIG. 2, an electric measuring unit 13, a processing unit 14, and a power supply unit 15 are also included and, in one embodiment, reside entirely within environmental sensing device interior/accommodating space 201 formed in environmental sensing device body 10.

Referring to FIG. 2, in one embodiment, electric measuring unit 13 measures current, voltage, capacitance, and temperature of the surrounding environment corresponding to the first conducting unit 11 and the second conducting unit 12.

In one embodiment, electric measuring unit 13 transmits a first signal including data indicating the measurement of the current, voltage, capacitance, and temperature of the environment.

In various embodiments, electric measuring unit 13 can be any electric measuring unit capable of collecting environmental data and transferring the data to processing unit 14, as discussed herein, and/or as known/available in the art at the time of filing, and/or as developed/made available after the time of filing.

In one embodiment, processing unit 14 is disposed entirely in the environmental sensing device interior/accommodating space 201 for receiving the first signal and calculating a moisture of the surrounding environment according to environmental data and the measurement of the current, voltage, capacitance, and temperature of the surrounding environment. In one embodiment, processing unit 14 is disposed in the environmental sensing device interior/accommodating space 201 and receives the first signal from electric measuring unit 13. In one embodiment, the processing unit 14 then processes the data to calculate the moisture of the surrounding environment according to environmental data and the measurements of current, voltage, capacitance, and temperature.

In one embodiment, the environmental data received by processing unit 14 includes the dielectric constant of soil or air at different moisture levels. In other words, the disclosed environmental sensing devices measure the moisture of the surrounding environment in real time. This can help manage the quality of the crops and also automate the process of doing so. For instance, the electric measuring unit 13 and the processing unit 14 may comprise, but are not limited to, an IC, a PLC or the like. In various other embodiments, processing unit 14 can be any processing unit capable of receiving data from electric measuring unit 13 and processing the data to calculate the moisture of the surrounding environment according to environmental data and the measurements of current, voltage, capacitance, and temperature, as discussed herein, and/or as known/available in the art at the time of filing, and/or as developed/made available after the time of filing.

In one embodiment, power supply unit 15 is disposed entirely in the environmental sensing device interior/accommodating space 201 for supplying power to electric measuring unit 13 and processing unit 14. In one embodiment, power supply unit 15 can be, for example, a battery or any suitable power supply. In various other embodiments, power supply unit 15 can be any power supply capable of providing power to electric measuring unit 13 and processing unit 14, as discussed herein, and/or as known/available in the art at the time of filing, and/or as developed/made available after the time of filing.

In various embodiments, in addition to electric measuring unit 13, processing unit 14, and power supply unit 15, the disclosed environmental sensing devices can also include other units disposed within environmental sensing device interior/accommodating space 201 for collecting and/or processing other types of data.

FIG. 3 shows a functional block diagram of the internal components of one embodiment of the disclosed environmental sensing devices.

Referring to FIGS. 1A, 1B, IC, 1D, 1E, 3, and 4A, as seen in FIG. 3, in one embodiment, the disclosed environmental sensing devices include a temperature sensing unit 16 disposed in the environmental sensing device interior/accommodating space 201 for sensing temperature of the surrounding environment.

In one embodiment, temperature sensing unit 16 transmits a second signal containing temperature data. In one embodiment, processing unit 14 receives the second signal and uses the second signal data to calibrate the measurement of the temperature of the surrounding environment according to the second signal, so as to obtain a more accurate temperature reading.

In one embodiment, the processing unit 14 calculates the moisture of the surrounding environment according to the environmental data, the measurement of the current, voltage, and capacitance of the surrounding environment and/or the calibrated temperature of the surrounding environment, etc. Because the capacitance is temperature dependent and moisture is affected by the temperature. The disclosed environmental sensing device can provide a more accurate and precise temperature reading of the surrounding environment by the calibration of the temperature sensing unit 16, resulting in a more reliable calculation of moisture than prior art systems can provide.

In various embodiments, temperature sensing unit 16 can be any temperature sensing unit capable of sensing temperature of the surrounding environment and transmitting a second signal as discussed herein, and/or as known/available in the art at the time of filing, and/or as developed/made available after the time of filing.

In one embodiment, the disclosed environmental sensing devices include a communication unit 17 disposed in the environmental sensing device interior/accommodating space and electrically connected to the processing unit for communicating with an external data processing device, such as a cellphone or personal computer.

In one embodiment, communication unit 17, including a data transmission device, is entirely disposed within the environmental sensing device interior/accommodating space. In various embodiments, communication unit 17 can be a WiFi communication unit, a LoRa communication unit, a Cellular communication unit, a Bluetooth communication unit, and/or any communication unit as discussed herein, and/or as available/known in the art at the time of filing, and/or as made

In one embodiment, communication unit 17 is electrically connected to processing unit 14 for wirelessly communicating with an external data processing device.

In one embodiment, communication unit 17 may be a wireless communication component disposed within environmental sensing device interior/accommodating space 201 using LTE technology (CAT-M1, NB-IoT), but it is not limited thereto.

In one embodiment, the disclosed environmental sensing devices exchange information with an external data processing device (such as a mobile device, a computer, a server, etc.) through communication unit 17.

In one embodiment, the disclosed environmental sensing devices transmit real-time information such as moisture levels and temperature to the external data processing device or receive environmental data from the external data processing device wirelessly using an internal data transmission device disposed entirely within environmental sensing device interior/accommodating space 201.

In various embodiments, communication unit 17 can be any communication unit 17 capable communicating with an external data processing device, such as a cellphone or personal computer, using an internal antenna/data transmission device housed entirely within environmental sensing device interior/accommodating space 201, as discussed herein, and/or as known/available in the art at the time of filing, and/or as developed/made available after the time of filing.

In one embodiment, the disclosed environmental sensing devices also include a positioning unit 18 disposed in the environmental sensing device interior/accommodating space 201 and electrically connected to processing unit 14 for tracking the location of the environmental sensing device, and therefore the crops with which the environmental sensing device resides, during the entire growth and processing cycles.

In one embodiment, positioning unit 18 is an electronic component such as a global positioning system (GPS) module, but it is not limited thereto. In one embodiment, the disclosed environmental sensing devices track the location of the environmental sensing device using the positioning unit 18 which can be useful in identifying conditions of the crops, such as whether the crops are currently at the stage of growing, transportation or storage, etc.

In one embodiment, communication unit 17 transmits a third signal indicating the location of the environmental sensing device to an external database (not shown). In one embodiment, the database includes pre-saved worldwide geographic information or meteorological information.

In one embodiment, the database sends the geographic information or meteorological information of the location of the environmental sensing device to communication unit 17 based on the data included in the third signal.

In one embodiment, processing unit 14 then calculates the moisture of the surrounding environment according to the environmental data, such as the measurement of the current, voltage, and capacitance of the surrounding environment and the calibrated temperature of the surrounding environment and the geographic information or meteorological information, etc.

Since soil moisture reacts differently in different soil types, the disclosed environmental sensing device can be pre-calibrated so as to correspond to different soil types, such as sand, silt, clay, etc. In one embodiment, the positioning unit 18 is used to determine the type of soil in which the environmental sensing device is disposed, by communicating with the database and the environmental sensing device can then automatically switch the soil moisture profile to match that of the soil in which the environmental sensing device, and therefore the crop, is currently disposed, so as to provide a more accurate moisture reading.

In various embodiments, positioning unit 18 can be any positioning unit capable of tracking the location of the environmental sensing device, as discussed herein, and/or as known/available in the art at the time of filing, and/or as developed/made available after the time of filing.

In one embodiment, the disclosed environmental sensing devices include a storage unit 19 disposed in the environmental sensing device interior/accommodating space 201 for receiving and recording the measurement of the current, voltage, capacitance, and temperature of the surrounding environment, the moisture calculated by the processing unit and the corresponding time.

In one embodiment, storage unit 19 receives and records the measurement of the current, voltage, capacitance, and temperature of the surrounding environment, the data of the moisture calculated by the processing unit 14, and the relative event/time log, etc. In one embodiment, storage unit 19 can be a memory, but it is not limited thereto. Environmental data such as changes of moisture through a predetermined period of time, can be stored and accessed in the storage unit 19. Further, the storage unit 19 may be used as a back-up memory to save the measurements and/or the calculated values by the disclosed environmental sensing device.

In various embodiments, storage unit 19 can be any storage unit 19 capable of receiving and recording the measurement of the current, voltage, capacitance, and temperature of the surrounding environment, the moisture calculated by the processing unit and the corresponding time, as discussed herein, and/or as known/available in the art at the time of filing, and/or as developed/made available after the time of filing.

In one embodiment, the disclosed environmental sensing device further includes an acceleration sensing unit 20 disposed in the environmental sensing device interior/accommodating space 201 and electrically connected to processing unit 14 for sensing an external force received by/imposed on environmental sensing device body 10.

In one embodiment, acceleration sensing unit 20 can be a 3-axis accelerometer, but it is not limited thereto. In one embodiment, since the disclosed environmental sensing devices are designed to remain with the crops at all stages of the crop processing cycle, including during harvesting, loading, transporting, unloading and storing acceleration, sensing unit 20 can sense the external forces received by/imposed on the crops during the entire crop processing cycle.

For instance, in one embodiment, the external forces may be incurred by other objects hitting the environmental sensing device or by the environmental sensing device hitting something else. Crops may be harvested using heavy machinery and pass through multiple pieces of machinery and locations, and as such there is a high potential for them to be dropped from excessive heights or at excessive speeds which can cause bruising to the crops. Bruising may reduce the quality and quantity of the crops and may further in turn create disease. This process can be monitored by the disclosed environmental sensing device, this data can then be used advise the farmers on how to better manage their harvesting and crop transportation operations. In other instances the data can be used to train ML and/or AI models, and update these models, to provide predictive data and again advise the farmers on how to better manage their harvesting and crop transportation operations. According to one embodiment, the environmental sensing device is rugged, waterproof, and has no external components extending more than 3 millimeters, to allow the users to harvest the crops without having to remove the disclosed environmental sensing device from the field.

In one embodiment, acceleration sensing unit 20 can be any acceleration sensing unit capable of sensing an external force received by/imposed on environmental sensing device body 10, as discussed herein, and/or as known/available in the art at the time of filing, and/or as developed/made available after the time of filing.

As noted above, FIG. 4A shows a specific illustrative example of a cross-sectional view of one embodiment of the disclosed environmental sensing devices.

As seen in FIG. 4A, in one embodiment, the disclosed environmental sensing devices can include a fixing frame 21 disposed in the environmental sensing device interior/accommodating space 201 for securing components disposed in the environmental sensing device interior/accommodating space 201. The fixing frame 21 may divide the environmental sensing device interior/accommodating space 201 into a plurality of one or more sub-cavities 211. Consequently, in one embodiment, fixing frame 21 may have one or more sub-cavities 211 illustrated in FIGS. 4A, 4B, 4C, 4D and 4E. The components of the environmental sensing device according to the present invention may then be fixed and secured in the cavities correspondingly.

Also shown in FIG. 4A is second conducting unit 12. As seen in FIG. 4A, in one embodiment, first conducting unit 11 (not shown in FIG. 4A, see FIGS. 1A through 1E And FIG. 5) and second conducting unit 12.

As discussed herein with respect to FIGS. 1A through 1E and FIG. 5, In one embodiment, the at least one first conducting unit 11 and at least one second conducting unit 12 are attached to environmental sensing device exterior surface 50 of the environmental sensing device body 10 such that the at least one first conducting unit and at least one second conducting unit extend a distance 57 no more than 3 millimeters from the exterior surface 50 of the environmental sensing device body 10.

In one embodiment, the at least one first conducting unit 11 and at least one second conducting unit 12 are attached to environmental sensing device exterior surface 50 of the environmental sensing device body 10 such that the at least one first conducting unit and at least one second conducting unit extend a distance 57 no more than 2 millimeters from the exterior surface 50 of the environmental sensing device body 10.

In one embodiment, the at least one first conducting unit 11 and at least one second conducting unit 12 are attached to environmental sensing device exterior surface 50 of the environmental sensing device body 10 such that the at least one first conducting unit 11 and at least one second conducting unit 12 extend a distance 57 no more than 1 millimeter from the exterior surface 50 of the environmental sensing device body 10.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 do not extend beyond environmental sensing device exterior surface 50 of the environmental sensing device body 10 at all and are recessed in environmental sensing device exterior surface 50 of the environmental sensing device body 10.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 are recessed below environmental sensing device exterior surface 50 of the environmental sensing device body 10.

In one embodiment, the disclosed environmental sensing devices may comprise one or more internal antennas (not shown) which may slide into their own independent cavities (not shown). These cavities for the antennas may be designed to be as far away from battery/power supply unit 15 as possible so as to avoid interference, in their own cavities to protect them and located there because that place may promote the most optimal signal strength.

FIGS. 4B, 4C, 4D, and 4E show various views of specific component placements in various one or more sub-cavities 211 of FIG. 4A, in accordance with one specific illustrative example of one embodiment.

In particular, FIG. 4B illustrates a cut away cross-sectional view of a disclosed environmental sensing device 400, and various components in accordance with one embodiment.

FIG. 4C illustrates a side cut away view of the disclosed environmental sensing device 400 and various components in accordance with one embodiment.

FIG. 4D illustrates a top cut away view of the disclosed environmental sensing device 400 and various components in accordance with one embodiment.

FIG. 4E illustrates a perspective diagonal cut away view of the disclosed environmental sensing device 400 and various components in accordance with one embodiment.

Referring now to FIGS. 4A, 4B, 4C, 4D and 4E together, as shown in FIGS. 4B, 4C, 4D and 4E, in one embodiment, environmental sensing device 400 includes environmental sensing device body 410 and environmental sensing device exterior surface 450. In addition, environmental sensing device 400 includes longitudinal length axis 455 and horizontal width axis 453.

As also shown in FIGS. 4B, 4C, 4D and 4E, in one embodiment, environmental sensing device 400 includes LTE antenna 401, GPS antenna 402, main processing unit board 403, ambient temperature and/or humidity sensor 404, moisture sensor 405, charging port 407, and battery power supply 409, each positioned within a respective sub-cavity of sub-cavities 211 of FIG. 4A.

In addition, FIG. 4D shows processing unit board 403 including Micro Controller Unit 411, Bluetooth Low Energy 413, Inertial Measurement Unit 415, and modem 417.

As a result of the structure and features shown in FIGS. 4A, 4B, 4C, 4D, and 4E, including the placement of the various components in respective sub-cavities 211 of FIG. 4A, moving or shaking the disclosed environmental sensing devices, such during harvesting, loading, unloading and transportation, will not cause damage to the internal components or reduce the efficacy of the disclosed environmental sensing devices. In other words, fixing frame 21 may protect the components from undesired conditions such as vibrations and drops that the environmental sensing device may incur to ensure continuous operation of the environmental sensing device.

The features of FIGS. 4A, 4B, 4C, 4D, and 4E combined with the fact that, as discussed above, in one embodiment, the only elements of the disclosed environmental sensing device that reside outside the environmental sensing device interior/accommodating space 201 are the at least one first conducting unit 11 and at least one second conducting unit 12 make the disclosed environmental sensing devices extremely sturdy and well suited for remaining with the crops at every stage of the growth and processing cycles.

As also discussed above, in one embodiment, the at least one first conducting unit 11 and at least one second conducting unit 11 are attached to, or recessed in, the exterior surface of the environmental sensing device body such that the at least one first conducting unit and at least one second conducting unit extend no more than 3 millimeters from the exterior surface of the environmental sensing device body.

Consequently, in one embodiment, the disclosed environmental sensing devices have no probes or other features that extend more than 3 millimeters from the environmental sensing device body and environmental sensing device exterior surface.

In addition, in one embodiment, the disclosed environmental sensing device body is either cylindrical, cuboid, spherical, or otherwise has a length dimension of the environmental sensing device body that is no more than twice a width or diameter dimension of the environmental sensing device.

Therefore, according to the disclosed embodiments, a fixing frame 21 increases reliability of the disclosed environmental sensing devices, the disclosed environmental sensing devices have no probes or other features that extend more than 3 millimeters from the environmental sensing device body and environmental sensing device exterior surface, and the disclosed environmental sensing device body is either cylindrical, spherical, cubic, and has a length dimension that is no more than twice a width or diameter dimension of the environmental sensing device. In addition, in one embodiment, environmental sensing device body 10 of the disclosed environmental sensing devices may include a plastic cover or cushion for the components to act as a further shield during any drops or impacts.

Thus, in contrast to the prior art, the combination of these and other features allow the disclosed environmental sensing devices to be buried with the crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops are they are transported, then remain with the crops as the crops loaded into storage units, and finally remain with the crops as the crops are stored; all using a single disclosed environmental sensing device.

As noted above, in one embodiment, the at least one first conducting unit 11 and at least one second conducting unit 12 are attached to environmental sensing device exterior surface 50 of the environmental sensing device body 10 such that the at least one first conducting unit and at least one second conducting unit extend no more than 3 millimeters from the exterior surface of the environmental sensing device body.

In one embodiment, the at least one first conducting unit 11 and at least one second conducting unit 12 are attached to environmental sensing device exterior surface 50 of the environmental sensing device body 10 such that the at least one first conducting unit and at least one second conducting unit extend no more than 2 millimeters from the exterior surface of the environmental sensing device body.

In one embodiment, the at least one first conducting unit 11 and at least one second conducting unit 12 are attached to environmental sensing device exterior surface 50 of the environmental sensing device body 10 such that the at least one first conducting unit 11 and at least one second conducting unit 12 extend no more than 1 millimeter from the exterior surface of the environmental sensing device body.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 do not extend outside environmental sensing device exterior surface 50 of the environmental sensing device body 10 are substantially flush with environmental sensing device exterior surface 50 of the environmental sensing device body 10.

In one embodiment, not shown, the at least one first conducting unit 11 and at least one second conducting unit 12 are recessed in environmental sensing device exterior surface 50 of the environmental sensing device body 10 such that the at least one first conducting unit 11 and at least one second conducting unit 12 are recessed below environmental sensing device exterior surface 50 of the environmental sensing device body 10.

FIG. 5 illustrates a schematic structural view of one illustrative example of one embodiment of the first conducting unit 11 and the second conducting unit 12.

As shown in FIG. 5, in one embodiment, the first conducting unit 11 is a two part annular first conducting unit including first conducting unit portions 11a and 11b to be disposed around environmental sensing device exterior surface 50 of the environmental sensing device body 10 as shown in FIGS. 1A, 1B, 1C, 1D, and 1E.

Similarly, as also shown in FIG. 5, in one embodiment, the second conducting unit 12 is a two part annular second conducting unit including second conducting unit portions 12a and 12b to be disposed around environmental sensing device exterior surface 50 of the environmental sensing device body 10 as shown in FIGS. 1A, 1B, 1C, 1D, and 1E.

Referring now to FIGS. 1A, 1B, 1C, 1D, 1E, 3, 4, and 5 together, in one embodiment, first conducting unit portions 11a, 11b and the second conducting portions 12a, 12b are designed in a shape of an arc or an arch, or any other shape and/or of any number or portions, in accordance with the shape of the body 10.

In one embodiment, the first conducting unit portions 11a, 11b and the second conducting unit portions 12a, 12b can be disposed around the outside of the body 10. It should be noted that, in the embodiment of FIG. 5, there are a set of two first conducting unit portions 11a, 11b and a set of two second conducting unit portions 12a, 12b. In this embodiment, the electric measuring unit 13 can sense measurements (including the current, the voltage, the capacitance and the temperature) of the two sets of the first conducting unit portions 11a, 11b and the second conducting unit portions 12a, 12b.

By these two sets of measurements, two measures of environmental moisture can be calculated by the environmental sensing device of the present invention; one is the environmental moisture corresponding to the positions of the first conducting unit portion 11a and the second conducting unit portion 12a, and the other is the environmental moisture corresponding to the positions of the first conducting unit portion 11b and the second conducting unit portion 12b.

In various embodiments, the number of the first conducting unit portions and the second conducting unit portions can be increased or decreased in sets as needed to sense the environmental moisture, as more moisture readings may provide a more accurate measurement of the environmental conditions.

In another embodiment (not shown), the disclosed environmental sensing device may comprise at least two first conducting units 11, at least two second conducting units 12 and at least two electric measuring units (not shown), such as electric measuring unit 13. Each of the electric measuring units may measure the current, the voltage, the capacitance and the temperature, etc., of each set of the first conducting units 11 and the second conducting units 12.

In another embodiment (not shown), the disclosed environmental sensing device may comprise two or more temperature sensing units, such as temperature sensing unit 16.

Regarding the abovementioned embodiments, the at least two conducting units and electric measuring units and the at least two temperature sensing units may be functionally independent (i.e. the conducting units, the electric measuring units and the temperature sensing units have no overlapping functions to each other) to better improve the accuracy and to prevent any data inconsistencies. The conducting units, the electric measuring units and the temperature sensing units are also omitted from accessing history data to prevent any discrepancies which might influence the correctness of data.

According to another embodiment (not shown), the environmental sensing device may further comprise a common moisture sensor which is functionally independent of the environmental sensing device. The common moisture sensor may be any moisture sensors known in the art. In this embodiment, the common moisture sensor may sense the moisture, which may be compared with the moisture calculated by the processing unit 14 for the users' reference.

In one embodiment, the first conducting unit 11 and the second conducting unit 12 can be made of conduction materials which are resistant to oxidization, such as stainless steel or the like, but they are not limited thereto. Since easily oxidized materials such as copper or copper plating material may degrade device performance or contaminate the crop and the soil.

As noted, in one embodiment, the first conducting unit 11 and the second conducting unit 12 may be made of stainless steel. It also ensures a longer life of the environmental sensing device of the present invention.

In one embodiment, the disclosed environmental sensing device may further conduct the various calculations with a noise filtering algorithm. For power consumption reasons, signals emitted by the first conducting unit and the second conducting unit may be low powered. In order to reduce the impact of background noise and unpredictable environmental surroundings, several techniques may be used in the present invention.

For example, to eliminate white noise type interference like thermal noise, the same signal may be sent multiple times and a low pass filter may be used. Environmental interferences are more unpredictable, and due to an object may responses differently on different frequency, multiple different frequencies are used and the results are compared to get a coherent trend. After the two stages, the number is kept in a storage buffer. Then filters, such as Kalman filter or the like, may be used to deprive the longer trend.

In one embodiment, once at least one of the disclosed environmental sensing devices discussed above with respect to FIGS. 1, 1B, 1C, 1D, 1E, 2, 3, 4, and 5 is provided at operation 603, process flow proceeds to operation 605.

In one embodiment at operation 605 the at least one disclosed environmental sensing device of operation 603 is buried in the soil in which crops grow.

In one embodiment, once the at least one disclosed environmental sensing device of operation 603 is buried in the soil in which crops grow at operation 605, process flow proceeds to operation 607.

In one embodiment, at operation 607, the at least one disclosed environmental sensing device of operations 603 and 605 buried in the soil with the crops remains in the soil with the crops as the crops grow and the at least one disclosed environmental sensing device of operations 603 and 605 is harvested together with the crops.

In one embodiment, once the at least one disclosed environmental sensing device of operations 603 and 605 is harvested together with the crops at 607, process flow proceeds to operation 609.

In one embodiment, at operation 609, the at least one disclosed environmental sensing device of operations 603, 605, and 607 remains with the crops as the crops are loaded into a transportation vehicle along and the at least one disclosed environmental sensing device of operations 603, 605, and 607 remains with the crops as the crops are transported to a storage facility.

In one embodiment, once the at least one disclosed environmental sensing device of operations 603, 605, and 607 remains with the crops as the crops are loaded into a transportation vehicle along and the at least one disclosed environmental sensing device of operations 603, 605, and 607 remains with the crops as the crops are transported to a storage facility at operation 609, process flow proceeds to operation 611.

In one embodiment, at operation 611, the at least one disclosed environmental sensing device of operations 603, 605, 607, and 609 remains with the crops and is unloaded into storage units at the storage facility along with the crops.

In one embodiment, once the at least one disclosed environmental sensing device of operations 603, 605, 607, and 609 remains with the crops and is unloaded into storage units at the storage facility along with the crops at operation 611, process flow proceeds to operation 613.

In one embodiment, at operation 613, the at least one disclosed environmental sensing device of operations 603, 605, 607, 609 and 611 remains with the crops in the storage units as the crops are stored.

In one embodiment, once the at least one disclosed environmental sensing device of operations 603, 605, 607, 609 and 611 remains with the crops in the storage units as the crops are stored at operation 613, process flow proceeds to end 630.

At each of operations 605, 607, 609, 611 and 613 the at least one disclosed environmental sensing device provided at operation 603 provides at least some of the various environmental and various other data discussed above with respect to with respect to FIGS. 1, 1B, 1C, 1D, 1E, 2, 3, 4, and 5.

As noted above this is possible because, in contrast to the prior art methods and systems, the only elements of the at least one disclosed environmental sensing device provided at operation 603 that reside outside the environmental sensing device interior/accommodating space are the at least one first conducting unit and at least one second conducting unit.

As also discussed above, in one embodiment, the at least one first conducting unit and at least one second conducting unit are attached to, or are recessed below, the exterior surface of the environmental sensing device body such that the at least one first conducting unit and at least one second conducting unit extend no more than 3 millimeters from the exterior surface of the environmental sensing device body.

Consequently, in one embodiment, the at least one disclosed environmental sensing device provided at operation 603 has no probes or other features that extend more than 3 millimeters from the environmental sensing device body and environmental sensing device exterior surface.

In addition, in one embodiment, the at least one disclosed environmental sensing device provided at operation 603 is either spherical, cubic, ellipsoidal, or has a length dimension of the environmental sensing device body is no more than twice a width or diameter dimension of the environmental sensing device.

As a result, the at least one disclosed environmental sensing device provided at operation 603 is rugged, waterproof, and has no exterior feature that extends more than 3 millimeters from the environmental sensing device body. Thus, in contrast to the prior art, the at least one disclosed environmental sensing device provided at operation 603 is designed to, and is capable of, being buried with the crops at operation 605 and remaining continuously with the crops during the entire lifetime of the crops of operations 607, 609, 611 and 613.

Consequently, in accordance with embodiments of the disclosed process 600 the disclosed environmental sensing devices can be buried with the crops at operation 605, then remain buried in the ground with the crops while they grow and remain with the crops and be harvested with the crops as they are harvested using mechanical/automated harvesting devices at operation 607, then remain with the crops and be loaded into transportation vehicles with the crops and then remain with the crops are they are transported at operation 609, then remain with the crops as the crops loaded into storage units at operation 611, and finally remain with the crops as the crops are stored at operation 613; all using the same disclosed environmental sensing device and while that same disclosed environmental sensing device provides the various environmental data described above.

Consequently, process 600 provides a technical solution to the long-standing technical problem of accurately collecting data regarding the environmental factors and forces a given crop is subjected to at each stage of the lifetime of the crops using a single type of environmental sensing device.

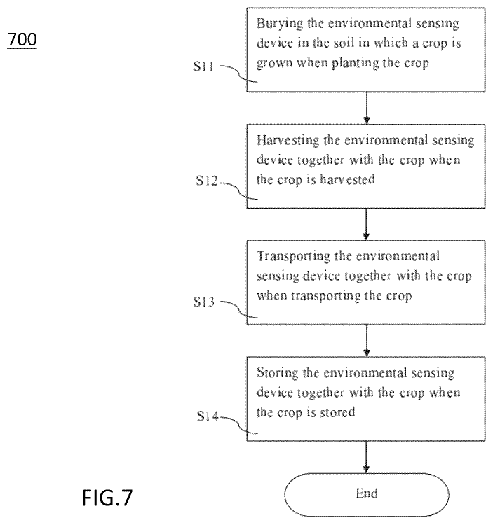

FIG. 7 is a flow chart of application method 700 of the disclosed environmental sensing devices according to one embodiment. Referring to FIG. 7.

The steps of the application method 700 of the disclosed environmental sensing device include:
S11. Burying the environmental sensing device, such as any disclosed environmental sensing device discussed above with respect to operation 603 of process 600, in the soil in which crops grow when planting the crops;
S12. Harvesting the environmental sensing device together with the crops when harvesting the crops;
S13. Transporting the environmental sensing device together with the crops when transporting the crops; and
S14. Storing the environmental sensing device together with the crops when the crops are stored.

In the application method 700 according to the present invention, the aforementioned environmental sensing device is disposed near the crops. In one embodiment, the dimension and shape of the environmental sensing device can be designed with reference to the crop (such as a potato) to be monitored. By doing so, the moisture and other environmental conditions sensed by the environmental sensing device are substantially the same as the actual environmental conditions around the crops.

In step S11, the disclosed environmental sensing device, such as any disclosed environmental sensing device discussed above with respect to operation 603 of process 600, may be buried in the soil close to the target crops, e.g. close to the root of the crops, or close to the planted seeds, etc. In another embodiment, multiple environmental sensing devices are buried at different depths respectively.

In step S12, the environmental sensing device can be harvested together with the crops by heavy machinery.

In steps S13 and S14, the environmental data of the processing unit 14 can be reset as the environment changes, so that the sensing result is kept accurate and relevant to different stages of the crops. In the application method of the disclosed environmental sensing device, environment conditions may differ at different stages of the crops. For instance, environmental conditions around the crops during transportation are likely to be different to those of the crops during growth. The environmental data can be reset and continuously monitored by the environmental sensing device when the crops changes from one environment to another.

Subsequently, environmental conditions such as temperature and humidity, etc., around the crops can then be managed as desired.

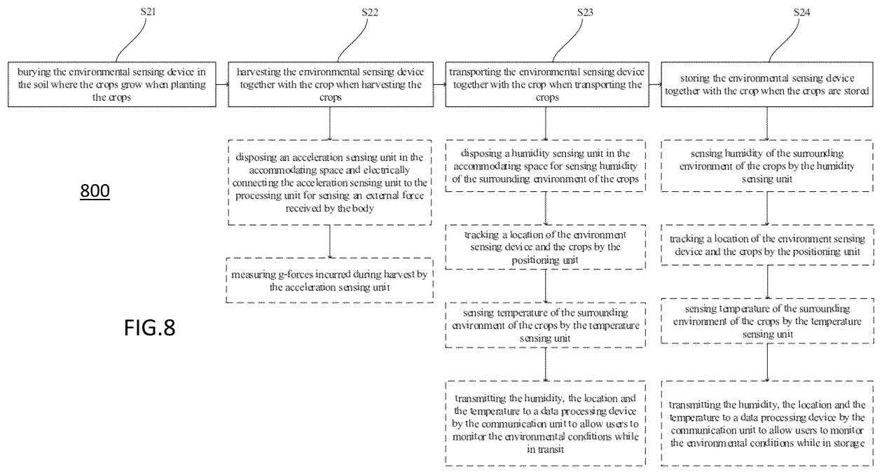

As seen in FIG. 8, the present invention further provides a method 800 for measuring environmental conditions around crops, comprising the following steps:

S21 burying the environmental sensing device such as any disclosed environmental sensing device discussed above with respect to operation 603 of process 600 in the soil where the crops grow when planting the crops. The environmental sensing device may comprise a body, at least one first conducting unit, at least one second conducting unit, an electric measuring unit, a communication unit, a positioning unit, a temperature sensing unit, a processing unit.

The body may comprise an environmental sensing device interior/accommodating space inside. The electric measuring unit, the communication unit, the positioning unit, the temperature sensing unit and the processing unit may be disposed in the environmental sensing device interior/accommodating space.

The at least one first conducting unit and the at least one second conducting unit may be disposed outside the body and the at least one second conducting unit may correspond to the first conducting unit; wherein the at least one first conducting unit and the at least one second conducting unit may contact the soil where the crops grow.

The electric measuring unit may be for measuring current, voltage, capacitance, and temperature, etc., of the surrounding environment corresponding to the first conducting unit and the second conducting unit; wherein the electric measuring unit further transmits a first signal according to the measurement of the current, voltage, capacitance, and temperature, etc., of the environment.

The communication unit may be electrically connected to the processing unit for wirelessly communicating with an external data processing device.

The positioning unit may be electrically connected to the processing unit for tracking a location of the environmental sensing device.

The temperature sensing unit may be for sensing temperature of the surrounding environment and transmitting a second signal.

The communication unit may transmit a third signal according to the location of the environmental sensing device to a database, wherein the database pre-saves worldwide geographic information and the database sends the geographic information of the location of the environmental sensing device to the communication unit. According to the geographic information from the database, the processing unit may switch the moisture profile to match the soil type that the environmental sensing device is geographically placed in. This may provide a more accurate moisture reading, since moisture behaves differently in different soil types.

The processing unit may receive the first signal and the second signal and calibrating the measurement of the temperature of the surround environment by the second signal; and the processing unit may calculate soil moisture of the soil where the crops are planted according to the environmental data, the measurement of the current, voltage, and capacitance of the surrounding environment, the calibrated temperature of the surrounding environment and the geographic information, etc.

In one embodiment, the method for measuring environmental conditions around crops may further comprise the following steps:

S22. harvesting the environmental sensing device together with the crop when harvesting the crops.

In step S22, the method for measuring environmental conditions around crops may further include disposing an acceleration sensing unit in the environmental sensing device interior/accommodating space and electrically connecting the acceleration sensing unit to the processing unit for sensing an external force received by the body and measuring g-forces incurred during harvest or transportation by the acceleration sensing unit. According to the present invention, the external forces may be incurred by other object hitting the environmental sensing device or otherwise by the environmental sensing device hitting something else.

In one embodiment, the method for measuring environmental conditions around crops may further comprise the following steps:

S23. transporting the environmental sensing device together with the crop when transporting the crops.

In step S23, the method for measuring environmental conditions around crops may further disposing a humidity sensing unit in the environmental sensing device interior/accommodating space for sensing humidity of the surrounding environment of the crops; and tracking a location of the environmental sensing device and the crops by the positioning unit; sensing temperature of the surrounding environment of the crops by the temperature sensing unit; sensing an external force received by the body and measuring g-forces incurred during transportation by the acceleration sensing unit; and transmitting the humidity, the location, the temperature and the external force and the g-force to an external data processing device by the communication unit to allow users to monitor the environmental conditions while the crops are in transit.

In one embodiment, the method for measuring environmental conditions around crops may further comprise the following steps:

S24. storing the environmental sensing device together with the crops when the crops are stored.

In step S24, the method for measuring the environmental condition around crops may include sensing humidity of the surrounding environment of the crops by the humidity sensing unit; tracking a location of the environmental sensing device and the crops by the positioning unit; sensing temperature of the surrounding environment of the crops by the temperature sensing unit; and transmitting the humidity, the location and the temperature to an external data processing device by the communication unit to allow users to monitor the environmental conditions while in storage.

The disclosed environmental sensing device comprises at least one first conducting unit and at least one second conducting unit disposed outside the body that extend no more than 3 millimeters from the exterior surface of the environmental sensing device. The environmental sensing device measures current, voltage, capacitance, and temperature of the surrounding environment corresponding to the first conducting unit and the second conducting unit by the electric measuring unit and calculates a moisture of the surrounding environment through the processing unit. Compared to prior art sensing devices, such as those operate based on osmotic balance, the moisture provided by the disclosed environmental sensing device is more accurate and reliable because it is calculated according to the capacitance combined with other environmental data and a unique noise filtering algorithm. Also, the disclosed environmental sensing device can provide real-time moisture levels to users which allows the users to precisely irrigate their crop accordingly, so inappropriate actions such as over watering or under watering, etc., can be prevented. Subsequently, utilizing the disclosed environmental sensing device to farm can be more economical as the consumed water, fuel or electricity, etc., can be effectively reduced. In addition, users may receive the in-field conditions wirelessly by environmental sensing device according to the present invention and may not need to visit every fields to evaluate the conditions physically, which would save labor, fuel and time, etc. This allows the users to grasp the status of all the farms, crops and/or relative operations, etc., with significant simplicity and efficiency.

Furthermore, the disclosed environmental sensing device provides reliability and robustness as the electronic components are substantially placed and covered fully in the environmental sensing device interior/accommodating space of the body. In addition, the disclosed environmental sensing device is able to continuously monitor the environmental conditions of the crops of different stages of planting, transport and storage, etc. The positioning unit of the disclosed environmental sensing device allows users to also track crops throughout the supply chain process, which improves accountability and traceability. In addition, the communication unit or other sensing components can be added, so more information can be collected to obtain environmental conditions with more accuracy, or more functions (e.g. communication) can be added to provide more flexible in applying the environmental sensing device.

The foregoing descriptions of the detailed embodiments are only illustrated to disclose the features and functions of the present invention and not restrictive of the scope of the present invention. It should be understood to those in the art that all modifications and variations according to the spirit and principle in the disclosure of the present invention should fall within the scope of the appended claims.

As shown above, disclosed herein are various embodiments of a method and system that provide a technical solution to the long-standing technical problem of accurately collecting data regarding the environmental factors and forces a given crop is subjected to at each stage of the lifetime of the crops using a single type of environmental sensing device.

To this end, disclosed above are methods and systems that provide an environmental sensing device that can be buried with the crops and stay with the crops during the entire lifetime of the crops, i.e., an environmental sensing device that can be buried with crops, then remain buried in the ground with the crops while they grow, then remain with the crops and be harvested with the crops as they are harvested using mechanical/automated harvesting devices, then remain with the crops and be loaded into transportation vehicles with the crops, then remain with the crops as they are transported, then remain with the crops as the crops loaded into storage units, and finally remain with the crops as the crops are stored.

Consequently, the disclosed methods and systems provide a technical solution to the long-standing technical problem of accurately collecting data regarding the environmental factors and forces a given crop is subjected to at each stage of the lifetime of the crops using a single type of environmental sensing device.

In one embodiment, an environmental sensing device includes an environmental sensing device body that forms an environmental sensing device interior/accommodating space, and an environmental sensing device exterior surface.

In one embodiment, an environmental sensing device includes at least one first conducting unit attached to at least part of the exterior surface of the environmental sensing device body such that no portion of the at least one first conducting unit extends more than 3 millimeters from the exterior surface of the environmental sensing device body.

In one embodiment, an environmental sensing device includes at least one second conducting unit attached to at least part of the exterior surface of the environmental sensing device body such that no portion of the at least one second conducting unit extends more than 3 millimeters from the exterior surface of the environmental sensing device body.

In one embodiment, an environmental sensing device includes an electric measuring unit disposed entirely within the environmental sensing device interior/accommodating space the electric measuring unit measuring current, voltage, capacitance, and temperature of a surrounding environment of the environmental sensing device corresponding to the first conducting unit and the second conducting unit, the electric measuring unit being capable of transmitting a first signal that includes data indicating the measurement of the current, voltage, capacitance, and temperature of the surrounding environment.

In one embodiment, an environmental sensing device includes a processing unit disposed entirely within the environmental sensing device interior/accommodating space, the processing unit being capable of receiving the first signal from the electric measuring unit and processing the first signal data to calculate a moisture of the surrounding environment according.

In one embodiment, an environmental sensing device includes a power supply unit disposed entirely within the environmental sensing device interior/accommodating space for supplying power to the electric measuring unit and the processing unit.

In one embodiment, an environmental sensing device includes a communication unit, including a data transmission device, the communication unit and data transmission device being entirely disposed within the environmental sensing device interior/accommodating space.

In one embodiment, an environmental sensing device includes an environmental sensing device body having a general shape such that a length dimension of the environmental sensing device body is no more than twice a width or diameter dimension of the environmental sensing device body.

In one embodiment, an environmental sensing device includes an environmental sensing device body having a general shape selected from the group of environmental sensing device body general shapes including: a substantially cylindrical shape with a substantially circular cross section; a substantially cylindrical shape with a substantially oval or elliptical cross section; a substantially ellipsoidal shape; a substantially cube shape; a substantially cuboid shape; a substantially spherical shape; and a substantially multi-faced shape having a multi-side cross section.

In one embodiment, an environmental sensing device includes a first conducting unit and a second conducting that do not extend beyond the exterior surface of the environmental sensing device body. In one embodiment, the first conducting unit and the second conducting unit are annular and disposed around the exterior surface of the environmental sensing device body.

In one embodiment, the environmental sensing device includes a temperature sensing unit disposed in the environmental sensing device interior/accommodating space for sensing temperature of the surrounding environment and transmitting a second signal.

In one embodiment, the environmental sensing device includes a processing unit further used to receive the second signal and calibrating the measurement of the temperature of the surrounding environment by the second signal.

In one embodiment, the environmental sensing device includes a processing unit further used to calculate the moisture of the surrounding environment according to the environmental data, the measurement of the current, voltage, and capacitance of the surrounding environment and the calibrated temperature of the surrounding environment.

In one embodiment, the environmental sensing device includes a positioning unit disposed in the environmental sensing device interior/accommodating space and electrically connected to the processing unit for tracking a location of the environmental sensing device.

In one embodiment, the environmental sensing device includes a storage unit disposed in the environmental sensing device interior/accommodating space for receiving and recording the measurement of the current, voltage, capacitance, and temperature of the surrounding environment, the moisture calculated by the processing unit and the corresponding time.

In one embodiment, the environmental sensing device includes an acceleration sensing unit disposed in the environmental sensing device interior/accommodating space and electrically connected to the processing unit for sensing an external force received by the body.

In one embodiment, a process for measuring environmental conditions around crops is disclosed. In one embodiment, the process includes providing an environmental sensing device, the environmental sensing device includes an environmental sensing device body that forms an environmental sensing device interior/accommodating space, and an environmental sensing device exterior surface.

In one embodiment, the environmental sensing device provided includes at least one first conducting unit attached to at least part of the exterior surface of the environmental sensing device body such that no portion of the at least one first conducting unit extends more than 3 millimeters from the exterior surface of the environmental sensing device body.

In one embodiment, the environmental sensing device provided includes at least one second conducting unit attached to at least part of the exterior surface of the environmental sensing device body such that no portion of the at least one second conducting unit extends more than 3 millimeters from the exterior surface of the environmental sensing device body.

In one embodiment, the environmental sensing device provided includes an electric measuring unit disposed entirely within the environmental sensing device interior/accommodating space, the electric measuring unit measuring current, voltage, capacitance, and temperature of a surrounding environment of the environmental sensing device corresponding to the first conducting unit and the second conducting unit, the electric measuring unit being capable of transmitting a first signal that includes data indicating the measurement of the current, voltage, capacitance, and temperature of the surrounding environment.

In one embodiment, the environmental sensing device provided includes a processing unit disposed entirely within the environmental sensing device interior/accommodating space, the processing unit being capable of receiving the first signal from the electric measuring unit and processing the first signal data to calculate a moisture of the surrounding environment according.

In one embodiment, the environmental sensing device provided includes a power supply unit disposed entirely within the environmental sensing device interior/accommodating space for supplying power to the electric measuring unit and the processing unit.

In one embodiment, the environmental sensing device provided includes a communication unit, including a data transmission device, the communication unit and data transmission device being entirely disposed within the environmental sensing device interior/accommodating space.

In one embodiment, the environmental sensing device provided includes an acceleration sensing unit disposed in entirely in the environmental sensing device interior/accommodating space for sensing external forces received by the environmental sensing device and measuring g-forces incurred by the environmental sensing device.

In one embodiment, the environmental sensing device provided includes a positioning unit disposed entirely within the environmental sensing device interior/accommodating space, the processing unit capable of collecting data indicating a position of the environmental sensing device.

In one embodiment, the environmental sensing device provided includes a humidity sensing unit disposed entirely within the environmental sensing device interior/accommodating space.

In one embodiment, the environmental sensing device provided includes an environmental sensing device body having a general shape such that a length dimension of the environmental sensing device body is no more than twice a width or diameter dimension of the environmental sensing device body.

In one embodiment, the process includes burying the environmental sensing device in soil in which crops grow and using the environmental sensing device to collect environmental data regarding the soil in which crops grow at the time of planting.

In one embodiment, the process includes leaving the environmental sensing device in soil in which the crops grow until the crops are harvested and using the environmental sensing device to collect environmental data regarding the soil in which crops grow during the crop growth period.

In one embodiment, the process includes leaving the environmental sensing device with the crops as the crops are harvested and harvesting the environmental sensing device with the crops and using the environmental sensing device to collect environmental data as the crops are harvested.

In one embodiment, the process includes disposing an acceleration sensing unit entirely in the environmental sensing device interior/accommodating space and electrically connecting the acceleration sensing unit to the processing unit for sensing an external forces received by the environmental sensing device and measuring g-forces incurred by the environmental sensing device as the as the environmental sensing device is harvested with the crops.

In one embodiment, the process includes leaving the environmental sensing device with the crops as the crops are loaded into transportation vehicles and using the environmental sensing device to collect environmental data as the crops are loaded.

In one embodiment, the process includes leaving the environmental sensing device with the crops and transporting the environmental sensing device together with the crops when transporting the crops to a storage facility and using the environmental sensing device to collect environmental data as the crops are the crops are transported.

In one embodiment, the process includes providing a positioning unit disposed entirely within the environmental sensing device interior/accommodating space, the processing unit capable of collecting data indicating a position of the environmental sensing device.

In one embodiment, the process includes tracking a location of the environmental sensing device and the crops using the positioning unit.

In one embodiment, the process includes leaving the environmental sensing device with the crops as the crops are unloaded into storage units at the storage facility and using the environmental sensing device to collect environmental data as the crops unloaded.

In one embodiment, the process includes leaving the environmental sensing device with the crops as the crops are stored in the storage units and using the environmental sensing device to collect environmental data while the crops stored.

In one embodiment, the process includes when harvesting, loading, and unloading the environmental sensing device together with the crops, using the acceleration sensing unit to record external forces received by environmental sensing device and g-forces incurred.

In one embodiment, the process includes transmitting the data representing the external force and the g-force to an external data processing device using the communication unit.

In one embodiment, the process includes when transporting and storing the environmental sensing device together with the crops using the humidity sensing unit to record the humidity of the surrounding environment of the crops.

In one embodiment, the process includes transmitting data representing the humidity, the location, and the temperature data to an external data processing device using the communication unit.

In one embodiment, the process includes using the environmental data collected by the environmental sensing device to a train a model to be employed with a Machine Learning (ML) or Artificial Intelligence (AI) system.

In one embodiment, the process includes using a Machine Learning (ML) or Artificial Intelligence (AI) system and trained machine learning model to improve crop farming processes.

It should be noted that the language used in the specification has been principally selected for readability, clarity and instructional purposes, and may not have been selected to delineate or circumscribe the inventive subject matter. Accordingly, the disclosure of the present invention is intended to be illustrative, but not limiting, of the scope of the invention, which is set forth in the claims below.

In the discussion above, certain aspects of one embodiment include process steps and/or operations described herein for illustrative purposes in a particular order and/or grouping. However, the particular order and/or grouping shown and discussed herein are illustrative only and not limiting. Those of skill in the art will recognize that other orders and/or grouping of the process steps and/or operations are possible and, in some embodiments, one or more of the process steps and/or operations discussed above can be combined and/or deleted. In addition, sections of one or more of the process steps and/or operations can be re-grouped as sections of one or more other of the process steps and/or operations discussed herein. Consequently, the particular order and/or grouping of the process steps and/or operations discussed herein do not limit the scope of the invention as claimed below.

In addition, the features shown in the figures are identified using a particular nomenclature for ease of description and understanding, but other nomenclature is often used in the art to identify equivalent features.

Therefore, numerous variations, whether explicitly provided for by the specification or implied by the specification or not, may be implemented by one of skill in the art in view of this disclosure.

What is claimed is:

1. An environmental sensing device comprising:
an environmental sensing device body that forms an environmental sensing device interior/accommodating space, and an environmental sensing device exterior surface;
at least one first conducting unit attached to at least part of the exterior surface of the environmental sensing device body such that no portion of the at least one first conducting unit extends more than 3 millimeters from the exterior surface of the environmental sensing device body;
at least one second conducting unit attached to at least part of the exterior surface of the environmental sensing device body such that no portion of the at least one second conducting unit extends more than 3 millimeters from the exterior surface of the environmental sensing device body;
an electric measuring unit disposed entirely within the environmental sensing device interior/accommodating space, the electric measuring unit measuring current, voltage, capacitance, and temperature of a surrounding environment of the environmental sensing device corresponding to the first conducting unit and the second conducting unit, the electric measuring unit being capable of transmitting a first signal that includes data indicating the measurement of the current, voltage, capacitance, and temperature of the surrounding environment;
a processing unit disposed entirely within the environmental sensing device interior/accommodating space, the processing unit being capable of receiving the first signal from the electric measuring unit and processing the first signal data to calculate a moisture of the surrounding environment;
a power supply unit disposed entirely within the environmental sensing device interior/accommodating space for supplying power to the electric measuring unit and the processing unit; and
a communication unit, the communication unit and data transmission device being entirely disposed within the environmental sensing device interior/accommodating space.

2. The environmental sensing device of claim 1 wherein the environmental sensing device body is a general shape such that a length dimension of the environmental sensing device body is no more than twice a width or diameter dimension of the environmental sensing device body.

3. The environmental sensing device of claim 2 wherein the environmental sensing device body is a general shape selected from the group of environmental sensing device body general shapes consisting of:
a substantially cylindrical shape with a substantially circular cross section;
a substantially cylindrical shape with a substantially oval or elliptical cross section;
a substantially ellipsoidal shape;
a substantially cube shape;
a substantially cuboid shape;
a substantially spherical shape; and
a substantially multi-faced shape having a multi-side cross section.

4. The environmental sensing device of claim 1 wherein the first conducting unit and second conducting unit do not extend beyond the exterior surface of the environmental sensing device body.

5. The environmental sensing device of claim 1 wherein the first conducting unit and the second conducting unit are annular and disposed around the exterior surface of the environmental sensing device body.

6. The environmental sensing device of claim 1, further comprising:
a temperature sensing unit disposed in the environmental sensing device interior/accommodating space for sensing temperature of the surrounding environment and transmitting a second signal,
wherein the processing unit is further used to receive the second signal and calibrate the measurement of the temperature of the surrounding environment by the second signal,
further wherein the processing unit further calculates the moisture of the surrounding environment according to the environmental data, the measurement of the current, voltage, and capacitance of the surrounding environment and the calibrated temperature of the surrounding environment.

7. The environmental sensing device of claim 1 further comprising:
a positioning unit disposed in the environmental sensing device interior/accommodating space and electrically connected to the processing unit for tracking a location of the environmental sensing device.

8. The environmental sensing device of claim 1, further comprising:
a storage unit disposed in the environmental sensing device interior/accommodating space for receiving and recording the measurement of the current, voltage, capacitance, and temperature of the surrounding environment, the moisture calculated by the processing unit, and the corresponding time.

9. The environmental sensing device of claim 1, further comprising:
an acceleration sensing unit disposed in the environmental sensing device interior/accommodating space and electrically connected to the processing unit for sensing an external force received by the environmental sensing device.

10. An application method of the environmental sensing device of claim 1, comprising:
burying the environmental sensing device in the soil in which crops grow when planting the crops;
harvesting the environmental sensing device together with the crop when harvesting the crops;
transporting the environmental sensing device together with the crops when transporting the crops; and
storing the environmental sensing device together with the crop when the crops are stored.

11. A process for measuring environmental conditions around crops, comprising:
providing an environmental sensing device, the environmental sensing device including:
an environmental sensing device body that forms an environmental sensing device interior/accommodating space and an environmental sensing device exterior surface;
at least one first conducting unit attached to at least part of the exterior surface of the environmental sensing device body such that no portion of the at least one first conducting unit extends more than 3 millimeters from the exterior surface of the environmental sensing device body;
at least one second conducting unit attached to at least part of the exterior surface of the environmental sensing device body such that no portion of the at least one second conducting unit extends more than 3 millimeters from the exterior surface of the environmental sensing device body;
an electric measuring unit disposed entirely within the environmental sensing device interior/accommodating space the electric measuring unit measuring current, voltage, capacitance, and temperature of a surrounding environment of the environmental sensing device corresponding to the first conducting unit and the second conducting unit, the electric measuring unit being capable of transmitting a first signal that includes data indicating the measurement of the current, voltage, capacitance, and temperature of the surrounding environment;
a processing unit disposed entirely within the environmental sensing device interior/accommodating space, the processing unit being capable of receiving the first signal from the electric measuring unit and processing the first signal data to calculate a moisture of the surrounding environment;
a power supply unit disposed entirely within the environmental sensing device interior/accommodating space for supplying power to the electric measuring unit and the processing unit; and
a communication unit, the communication unit and data transmission device being entirely disposed within the environmental sensing device interior/accommodating space;
burying the environmental sensing device in soil in which crops grow and using the environmental sensing device to collect environmental data regarding the soil in which crops grow at the time of planting;
leaving the environmental sensing device in soil in which the crops grow until the crops are harvested and using the environmental sensing device to collect environmental data regarding the soil in which crops grow during the crop growth period; and
leaving the environmental sensing device with the crops as the crops are harvested and harvesting the environmental sensing device with the crops and using the environmental sensing device to collect environmental data as the crops are harvested.

12. The process for measuring environmental conditions around crops of claim 11, further comprising:
disposing an acceleration sensing unit entirely in the environmental sensing device interior/accommodating space and electrically connecting the acceleration sensing unit to the processing unit for sensing external forces received by the environmental sensing device and measuring g-forces incurred by the environmental sensing device as the as the environmental sensing device is harvested with the crops.

13. The process for measuring environmental conditions around crops of claim 11, further comprising:
leaving the environmental sensing device with the crops as the crops are loaded into transportation vehicles and using the environmental sensing device to collect environmental data as the crops are loaded; and
leaving the environmental sensing device with the crops and transporting the environmental sensing device together with the crops when transporting the crops to a storage facility and using the environmental sensing device to collect environmental data as the crops are the crops are transported.

14. The process for measuring environmental conditions around crops of claim 11, further comprising:
providing a positioning unit disposed entirely within the environmental sensing device interior/accommodating space, the positioning unit capable of collecting data indicating a position of the environmental sensing device; and
tracking a location of the environmental sensing device and the crops using the positioning unit.

15. The process for measuring environmental conditions around crops of claim 13, further comprising:
leaving the environmental sensing device with the crops as the crops are unloaded into storage units at a storage facility and using the environmental sensing device to collect environmental data as the crops unloaded; and
leaving the environmental sensing device with the crops as the crops are stored in the storage units and using the environmental sensing device to collect environmental data while the crops stored.

16. A process comprising:
providing an environmental sensing device, the environmental sensing device including:
an environmental sensing device body that forms an environmental sensing device interior/accommodating space, and an environmental sensing device exterior surface;
at least one first conducting unit attached to at least part of the exterior surface of the environmental sensing device body such that no portion of the at least one first conducting unit extends more than 3 millimeters from the exterior surface of the environmental sensing device body;
at least one second conducting unit attached to at least part of the exterior surface of the environmental sensing device body such that no portion of the at least one second conducting unit extends more than 3 millimeters from the exterior surface of the environmental sensing device body;
an electric measuring unit disposed entirely within the environmental sensing device interior/accommodating space the electric measuring unit measuring current, voltage, capacitance, and temperature of a surrounding environment of the environmental sensing device corresponding to the first conducting unit and the second conducting unit, the electric measuring unit being capable of transmitting a first signal that includes data indicating the measurement of the current, voltage, capacitance, and temperature of the surrounding environment;
a processing unit disposed entirely within the environmental sensing device interior/accommodating space, the processing unit being capable of receiving the first signal from the electric measuring unit and processing the first signal data to calculate moisture of the surrounding environment according;
a power supply unit disposed entirely within the environmental sensing device interior/accommodating space for supplying power to the electric measuring unit and the processing unit;
a communication unit the communication unit being entirely disposed within the environmental sensing device interior/accommodating space;
an acceleration sensing unit disposed in entirely in the environmental sensing device interior/accommodating space for sensing an external forces received by the environmental sensing device and measuring g-forces incurred by the environmental sensing device;
a humidity sensing unit disposed entirely within the environmental sensing device interior/accommodating space; and
a positioning unit disposed entirely within the environmental sensing device interior/accommodating space, the positioning unit capable of collecting data indicating a position of the environmental sensing device;
burying the environmental sensing device in soil in which crops grow and using the environmental sensing device to collect environmental data regarding the soil in which crops grow at the time of planting;
leaving the environmental sensing device in soil in which the crops grow until the crops are harvested and using the environmental sensing device to collect environmental data regarding the soil in which crops grow during the crop growth period;
leaving the environmental sensing device with the crops as the crops are harvested and harvesting the environmental sensing device with the crops and using the environmental sensing device to collect environmental data as the crops are harvested;
leaving the environmental sensing device with the crops as the crops are loaded into transportation vehicles and using the environmental sensing device to collect environmental data as the crops are loaded; and
leaving the environmental sensing device with the crops and transporting the environmental sensing device together with the crops when transporting the crops to a storage facility and using the environmental sensing device to collect environmental data as the crops are the crops are transported;
tracking a location of the environmental sensing device and the crops using the positioning unit;
leaving the environmental sensing device with the crops as the crops are unloaded into storage units at the storage facility and using the environmental sensing device to collect environmental data as the crops unloaded; and
leaving the environmental sensing device with the crops as the crops are stored in the storage units and using the environmental sensing device to collect environmental data while the crops stored.

17. The process of claim 16, further comprising:
when harvesting, loading, and unloading the environmental sensing device together with the crops using the acceleration sensing unit to record external forces received by environmental sensing device and g-forces incurred; and
transmitting the data representing the external force and the g-force to an external data processing device using the communication unit.

18. The process of claim 16, further comprising:
when transporting and storing the environmental sensing device together with the crops using the humidity sensing unit to record the humidity of the surrounding environment of the crops; and
transmitting data representing the humidity, the location, and the temperature data to an external data processing device using the communication unit.

19. The process of claim 16, further comprising:
using the environmental data collected by the environmental sensing device to train a model to be employed with a Machine Learning (ML) or Artificial Intelligence (AI) system.

20. The process of claim 16, further comprising:
using the Machine Learning (ML) or Artificial Intelligence (AI) system and trained machine learning model to improve crop farming processes.
